(19) United States
(12) Patent Application Publication
(10) Pub. No.: US 2018/0055486 A1
(43) Pub. Date: Mar. 1, 2018

(54) ULTRASOUND SIGNAL PROCESSING DEVICE, ULTRASOUND DIAGNOSTIC DEVICE, AND ULTRASOUND SIGNAL PROCESSING METHOD

(71) Applicant: Konica Minolta, Inc., Tokyo (JP)

(72) Inventor: Mineo TSUSHIMA, Souraku-gun (JP)

(21) Appl. No.: 15/669,469

(22) Filed: Aug. 4, 2017

(30) Foreign Application Priority Data
Aug. 23, 2016 (JP) 2016-162682

(51) Int. Cl. A61B 8/08; A61B 8/14; A61B 8/00

(52) U.S. Cl. CPC A61B 8/5207; A61B 8/4477; A61B 8/14

(57) ABSTRACT

Ultrasound signal processing device performing transmission events of transmitting converging ultrasound beams to subject using ultrasound probe having transducer elements, receiving ultrasound reflection from the subject, generating acoustic line signal, and including ultrasound signal processing circuitry configured to operate as: transmitter varying focal point defining position where ultrasound beams converge between transmission events and performing each transmission event; receiver generating, for each transmission event, receive signal sequences for the transducer elements based on ultrasound reflection; and delay-and-sum calculator generating acoustic line signal for each measurement point by performing processing for specifying measurement point signal for each transmission event and performing weighted delay-and-summing of measurement point signals for the transmission events, the processing including identification of transducer element on straight line passing through the measurement point and the focal point and specification of, as the measurement point signal, receive signal for the transducer element from the sequence for the transducer element.

Japanese Patent Application No. 2016-162682 filed on Aug. 23, 2016, including description, claims, drawings, and abstract, is incorporated herein by reference in its entirety.

### BACKGROUND

### Technological Field

The present invention is related to an ultrasound signal processing device, and an ultrasound diagnostic device equipped with the ultrasound signal processing device. In particular, the present invention relates to receive beam forming in an ultrasound signal processing device.

### Description of the Related Art

Typically, an ultrasound diagnostic device transmits ultrasound towards the inside of a subject via an ultrasound probe (referred to in the following as a “probe”), and receives reflected ultrasound (an echo) via the probe. The reflected ultrasound is generated within the subject due to tissues in the subject having different acoustic impedances. Further, an ultrasound diagnostic device generates an ultrasound tomographic image based on electric signals acquired through the reception of the reflected ultrasound, and displays the ultrasound tomographic image. An ultrasound tomographic image shows the structures of tissues inside the subject. Ultrasound diagnostic devices are widely used for the imaging diagnosis of subjects, for having low invasiveness and achieving real-time observation of tissues through tomographic images and the like.

A typical method conventionally applied for forming signals based on received reflected ultrasound (i.e., receive beam forming) is delay-and-sum beam forming. One example of delay-and-sum beam forming can be found disclosed in pages 42-45 of “Ultrasound Diagnostic Equipment”, written by Masayasu Itou and Tsuyoshi Mochizuki and published by Corona Publishing Co., Ltd (Aug. 26, 2002). According to this method, transmission beam forming (i.e., transmission of ultrasound towards the inside of the subject) is typically performed such that a transmitted ultrasound beam converges (focuses) at a predetermined focal depth inside the subject. Further, according to this method, measurement points are always set along the central axis of the transmitted ultrasound beam or a focal area. Due to this, the number of measurement points is small relative to the size of an ultrasound main irradiation area, and thus reflected ultrasound is not utilized in an efficient manner. In addition, with this method, it is also problematic that an acoustic line signal acquired from a measurement point distant from the transmission focal point has low spatial resolution and low S/N ratio. Note that in the present disclosure, an ultrasound main irradiation area indicates an area where an ultrasound beam propagates.

Meanwhile, a receive beam forming method is being proposed that utilizes a so-called synthetic aperture method to yield images with high spatial resolution and high quality not only from near the transmission focal point but also from areas other than near the transmission focal point. One example of receive beam forming utilizing the synthetic aperture method can be found disclosed in pages 395 through 405 of “Virtual Ultrasound Sources in High Resolution Ultrasound Imaging”, S. I. Nikolov and J. A. Jensen, in Proc, SPIE—Progress in Biomedical Optics and Imaging, Vol. 3, 2002. According to this method, delaying is performed taking into consideration both a propagation path of ultrasound and the time amount required for reflected ultrasound to arrive at a transducer element by travelling along the propagation path. Thus, the method achieves receive beam forming making use of not only reflected ultrasound from an area of an ultrasound main irradiation area near the transmission focal point but also reflected ultrasound from areas of the ultrasound main irradiation area other than the area near the transmission focal point. Due to this, the method enables generating acoustic line signals (signals generated based on ultrasound reflection from measurement points by receive beam forming) covering the entire ultrasound main irradiation area as well as the central axis of a transmitted ultrasound beam. In addition, the synthetic aperture method enables setting a virtual transmission focal point with respect to each measurement point based on multiple receive signals acquired for the same measurement point through multiple transmission events. Thus, the synthetic aperture method enables acquiring an ultrasound image with higher spatial resolution and higher S/N ratio than the receive beam forming method disclosed in “Ultrasound Diagnostic Equipment”.

### SUMMARY

Meanwhile, in the synthetic aperture method, for efficient use of ultrasound and high resolution, it is preferable that an area for which acoustic line signals for a single transmission event are generated (referred to in the following as a target area) have large size, and it is further preferable that the entire ultrasound main irradiation area be used as the target area. However, an increase in target area size brings about a proportional increase in the number of measurement points (computation target points for receive beam forming) in the target area and an increase in computation amount for delay-and-summing taking into consideration transmission and reception delays. Due to this, an increase in target area size necessitates hardware with high computation capability to achieve high-speed delay-and-sum computation, and thus gives rise to a problem of increased ultrasound diagnostic device cost. However, reduction in the number of measurement points for reducing the computation amount results in a decrease in resolution and S/N ratio.

The present invention has been made in view of the problems described above, and aims to provide an ultrasound signal processing device that enables greatly reducing computation amount in a synthetic aperture method utilizing converging-type transmission beam forming while enjoying an effect achieved by the synthetic aperture method of an improvement in spatial resolution and S/N ratio, and an ultrasound diagnostic device including the ultrasound signal processing device.

One aspect of the present invention is an ultrasound signal processing device that performs multiple transmission events of transmitting converging ultrasound beams to a subject by using an ultrasound probe having multiple transducer elements, and that receives ultrasound reflection from the subject for each of the transmission events to generate sequences of receive signals, and synthesizes sequences of receive signals generated for the respective transmission events to generate an acoustic line signal, the ultrasound signal processing device comprising ultrasound signal processing circuitry configured to operate as: a transmitter that varies a focal point defining a position where ultrasound beams converge between a plurality of transmission events and performs each of the transmission events by causing the ultrasound probe to transmit ultrasound beams directed to an inside of the subject; a receiver that generates, for each of the transmission events, sequences of receive signals for the respective transducer elements of the ultrasound probe based on ultrasound reflection that the ultrasound probe receives from a target area of the subject; and a delay-and-sum calculator that generates an acoustic line signal with respect to each of measurement points of the target area, by performing processing for specifying one measurement point signal for each of the transmission events and performing weighted delay-and-summing of measurement point signals specified for the respective transmission events, the processing including identification of one of the transducer elements that is located on a straight line passing through the measurement point and the focal point and specification of, as the measurement point signal, a receive signal corresponding to the identified transducer element from among the sequence of receive signals for the identified transducer element.

The ultrasound signal processing device pertaining to one aspect of the present invention and an ultrasound diagnostic device including the ultrasound signal processing device are capable of greatly reducing computation amount by refraining from performing delay-and-summing for each transmission event while enjoying an effect of an improvement in spatial resolution and signal S/N ratio achieved by virtual transmission focusing according to the synthetic aperture method. Note that weighted delay-and-summing indicates that weighting is substantially performed by only summing. With this configuration, it is possible to acquire preferable acoustic line signals without further performing weighting computation.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a functional block diagram illustrating the structure of an ultrasound diagnostic device 100 pertaining to embodiment 1;

FIG. 2 is a schematic illustrating a propagation path of ultrasound transmitted from a transmission beam former 103 pertaining to embodiment 1;

FIG. 3 is a functional block diagram illustrating the structure of a receive beam former 104 pertaining to embodiment 1;

FIG. 4 is a schematic illustrating a relationship between a target area Bx and a receive transducer element Rij corresponding to a measurement point Pij pertaining to embodiment 1;

FIG. 5A is a schematic pertaining to embodiment 1, illustrating one propagation path of ultrasound that is transmitted from the transmission aperture Tx and arrives at a receive transducer element Rij via a measurement point Pij; FIG. 5B is a schematic pertaining to embodiment 1, illustrating another propagation path of ultrasound that is transmitted from the transmission aperture Tx and arrives at a receive transducer element Rij via a measurement point Pij;

FIG. 6 is a schematic illustrating processing by an adder 1048 pertaining to embodiment 1 for generating an acoustic line signal by performing delay-and-summing of measurement point signals;

FIGS. 7A and 7B are schematics pertaining to embodiment 1, providing an overview of maximum overlap counts of acoustic line signals and amplification by an amplifier 1049;

FIG. 8 is a flowchart illustrating beam forming by the receive beam former 104 pertaining to embodiment 1;

FIG. 9 is a flowchart illustrating operations of the receive beam former 104 pertaining to embodiment 1 for specifying a measurement point signal for a measurement point Pij;

FIG. 10 is a schematic for explaining the operations of the receive beam former 104 pertaining to embodiment 1 for generating an acoustic line signal for a measurement point Pij;

FIG. 11 is a schematic illustrating a relationship between a target area Bx, and a receive transducer element Rij and a fixed receive transducer element Xij for a measurement point Pij pertaining to modification 1;

FIG. 12 is a flowchart illustrating beam forming by a receive beam former 104 of an ultrasound diagnostic device pertaining to modification 1;

FIG. 13 is a flowchart illustrating specification of measurement point signals for a measurement point Pij by the receive beam former 104 pertaining to modification 1;

FIG. 14 is a schematic for explaining the operations of the receive beam former 104 pertaining to modification 1 for generating an acoustic line signal for a measurement point Pij;

FIG. 15 is a functional block diagram illustrating the structure of a receive beam former 204 pertaining to embodiment 2;

FIG. 16 is a functional block diagram illustrating the structure of a second delay-and-sum calculator 2041 pertaining to embodiment 2;

FIG. 17A through FIG. 17C are schematics illustrating a target area Cx pertaining to embodiment 2;

FIG. 18 is a schematic illustrating a relationship between a receive aperture Rx set by a receive aperture setter pertaining to embodiment 2 and a transmission aperture Tx;

FIG. 19 is a flowchart illustrating beam forming by the receive beam former 204 pertaining to embodiment 2;

FIG. 20 is a flowchart illustrating first beam forming by a delay-and-sum calculator 1041 pertaining to embodiment 2;

FIG. 21 is a flowchart illustrating second beam forming by the second delay-and-sum calculator 2041 pertaining to embodiment 2;

FIG. 22 is a flowchart illustrating acoustic line signal generation for a measurement point Qmn by the second delay-and-sum calculator 2041 pertaining to embodiment 2;

FIG. 23A through FIG. 23C are schematics illustrating a target area Cx pertaining to modification 2 and a propagation path of ultrasound arriving at a receive transducer element from a transmission aperture via a measurement point Qmn;

FIG. 24A through FIG. 24D are ultrasound images acquired by receive beam forming in embodiment 1, modification 1, comparative examples 1 and 2, respectively.

### DETAILED DESCRIPTION OF EMBODIMENTS

Hereinafter, one or more embodiments of the present invention will be described with reference to the drawings. However, the scope of the invention is not limited to the disclosed embodiments.

### <How Inventor Arrived at Aspects of Present Invention>

The inventor has conducted various considerations for reducing computation amount while suppressing a decrease in spatial resolution and S/N ratio of acoustic line signals (referred to in the following as acoustic line signal quality) in an ultrasound diagnostic device deploying a synthetic aperture method.

Typically, converging-type transmission beam forming is performed by causing a wavefront to converge so that an ultrasound beam focuses at a certain depth of a subject (referred to in the following as a focal depth). In each transmission of ultrasound (transmission event), transducer elements that are used for ultrasound transmission (referred to in the following as a transmission transducer element array) mainly transmit ultrasound to the ultrasound main irradiation area. For example, when ultrasound transmission is performed with one measurement point set as the transmission focal point, the ultrasound main irradiation area has an hourglass shape, the bottom edge (i.e., base) of the ultrasound main irradiation area corresponds to the transmission transducer element array, and two straight lines each extending from a different end of the base towards the transmission focal point partition the ultrasound main irradiation area from the outside thereof Further, the wavefront of ultrasound transmitted from the transmission transducer element array forms an arc, being a segment of a circle whose center corresponds to the transmission focal point. Here, it should be noted that ultrasound beams do not always converge (i.e., focus) to a single point as described above. For example, ultrasound beams may converge to an area having a width corresponding to 1.5 times the width of a single transducer element to several times the width of a single transducer element. When ultrasound beams converge at such an area, the width of the ultrasound main irradiation area in a direction in which transducer elements are arrayed (referred to in the following as a transducer element array direction) decreases as approaching the transmission focal depth, equals the width of the transmission focal area in the transducer element array direction at the transmission focal depth, and increases in the transducer element array direction once again as departing the transmission focal depth towards deeper areas. For convenience of description, a center point of the focal area at the focal depth in such a case is referred to as a focal point. That is, regardless of whether or not ultrasound beams focus at a single point, the ultrasound main irradiation area converges, at the focal depth, at the focal point or at the focal area, which is an area including the focal point and the vicinity of the focal point. Meanwhile, at depths other than the focal depth, the greater the distance from the focal depth, the greater the width of the ultrasound main irradiation area in the transducer element array direction.

Further, with the synthetic aperture method, for each transmission event, measurement points can be set to cover the entire ultrasound main irradiation area of the transmission event. As such, it is preferable that the entirety of the ultrasound main irradiation area be set as a target area. Meanwhile, a target area for one transmission event cannot cover the entirety of an area corresponding to one frame image (referred to in the following as a region of interest (ROI)). As such, a plurality of transmission events, for each of which a different target area is set, need to be conducted to generate one frame ultrasound image. Taking this into consideration, for efficient use of ultrasound, it is preferable that a target area for a single transmission event cover as great an area of an ultrasound main irradiation area for the transmission event as possible. Further, in general, to improve spatial resolution and signal S/N ratio, it is preferable that target areas for two consecutive transmission events overlap one another as much as possible.

However, the number of measurement points included in a target area, that is, the number of computation target points for receive beam forming is proportional to target area size. Consequently, computation amount for delay-and-summing and the memory amount necessary to store acoustic line signals produced through the delay-and-summing are proportional to target area size. Due to this, an increase in target area size directly results in an increase in computation amount and memory amount required for ultrasound diagnostic devices. Further, when ultrasound diagnostic device computation capability is insufficient with respect to delay-and-summing computation amount, a decrease in temporal resolution may occur, and thus a decrease in usability may occur. This is because ultrasound diagnostic devices are not capable of achieving frame rate higher than that corresponding to their computation capability, and thus a decrease in ultrasound image frame rate may occur. Accordingly, in order to suppress such decrease in temporal resolution and usability, a processor with computation capability high enough to perform delay-and-summing computation at high speed, such as a high performance GPU, becomes necessary, which leads to an increase in ultrasound diagnostic device cost.

One measure that can be considered for reducing computation amount is reducing the number of measurement points included in the target area. However, a thoughtless reduction in the number of measurement points for reducing computation amount results in a decrease in resolution of ultrasound images and S/N ratio in accordance with a reduction in computation amount. Further, it is sometimes difficult to drastically reduce the computation amount just by reducing the number of measurement points. In view of this, in order to reduce computation amount while enjoying advantages of the synthetic aperture method, the inventor has considered a method of performing delay-and-summing in synthesizing for different transmission events instead of performing delay-and-summing for each transmission event. According to this method, it is possible to drastically reduce computation amount of delay-and-summing as disclosed in Japanese Patent Application Publication No. 2008-536578 for example. Hence, the inventor has sought for the advantages of the synthetic aperture method, that is, a method of suppressing degradation of acoustic line signal quality, and has arrived at an idea of identifying, with respect to each measurement point, a transducer element that is located on a straight line passing through the measurement point and a transmission focal point for each transmission event, and performing delay-and-summing of a receive signal sequence received by the identified transducer element. Due to this, it is possible to generate an acoustic line signal with respect to each measurement point based on receive signal sequences that are acquired while varying the position by associating the transmission focal point with the receive transducer element. This increases diversity of an ultrasound beam travel direction and a positional relationship between the measurement point and the receive transducer element, thereby suppressing a decrease in spatial resolution and signal S/N by virtual transmission beam forming. Further, it is possible to prevent acoustic line signals from being influenced by variation in properties (especially receive sensitivity) of transducer elements. This is because a different receive transducer element corresponds to one measurement point for each transmission event, in other words, a receive transducer element corresponding to one measurement point is not fixed for a plurality of transmission events.

The following embodiments each describe an ultrasound signal processing method and an ultrasound diagnostic device including the ultrasound signal processing method in detail, with reference to the accompanying drawings.

### Embodiment 1

### <Overall Structure>

The following describes an ultrasound diagnostic device 100 pertaining to embodiment 1, with reference to the accompanying drawings.

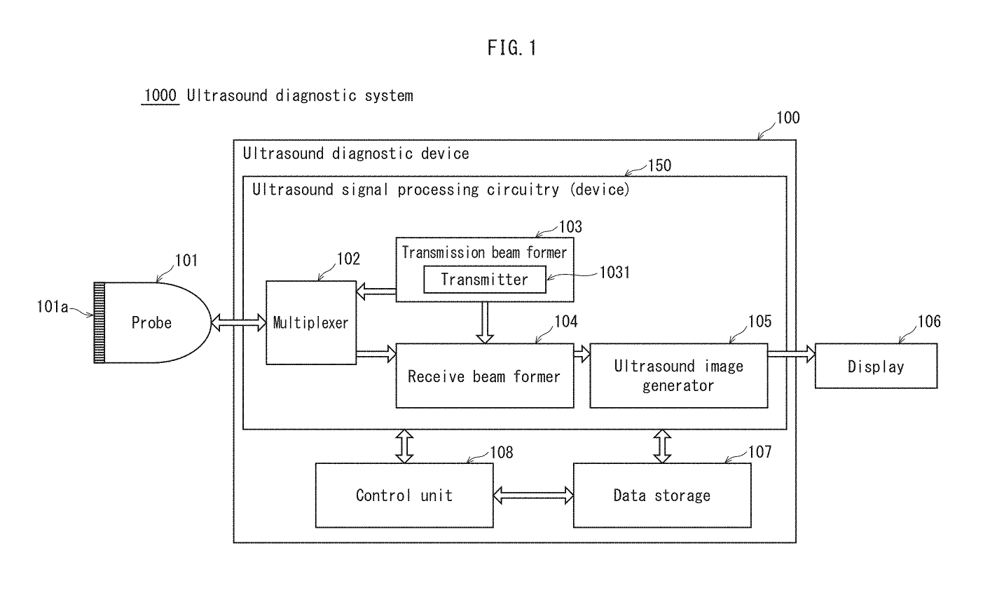

FIG. 1 illustrates functional blocks of an ultrasound diagnostic system 1000 pertaining to embodiment 1. As illustrated in FIG. 1, the ultrasound diagnostic system 1000 includes: a probe 101; the ultrasound diagnostic device 100; and a display unit 106. The probe 101 includes a plurality of transducer elements 101a. Each of the transducer elements 101a is capable of transmitting ultrasound towards the subject and receiving reflected ultrasound (echo signals). The ultrasound diagnostic device 100 causes the probe 101 to perform transmission/reception of ultrasound, and generates an ultrasound image based on signals output from the probe 101. The display unit 106 displays the ultrasound image on any display device provided thereto. The probe 101 and the display unit 106 are separately connectable to the ultrasound diagnostic device 100. FIG. 1 illustrates the ultrasound diagnostic device 100 with the probe 101 and the display unit 106 connected thereto. Alternatively, the ultrasound diagnostic device 100 may include therein the probe 101 and the display unit 106.

### <Structure of Ultrasound Diagnostic Device 100>

The ultrasound diagnostic device 100 includes a multiplexer 102; a transmission beam former 103; and a receive beam former 104. The multiplexer 102 selects one or more of the transducer elements 101a for ultrasound transmission and one or more of the transducer elements 101a for ultrasound reception. The multiplexer 102 may select different ones of the transducer elements 101a for ultrasound transmission and ultrasound reception. Further, the multiplexer 102 provides the transducer elements 101a for ultrasound transmission with input, and receives output from the transducer elements 101a for ultrasound reception. The transmission beam former 103 controls timings of application of a high voltage for ultrasound transmission to each of the transducer elements 101a for ultrasound transmission. The receive beam former 104 performs some amplification and A/D conversion on electric signals yielded by the transducer elements 101a for ultrasound reception, based on reflected ultrasound received by the probe 101, and performs receive beam forming to generate acoustic line signals. In addition, the ultrasound diagnostic device 100 includes an ultrasound image generator 105; a data storage 107; and a control unit 108. The ultrasound image generator 105 generates an ultrasound image (a B-mode image) based on signals output from the receive beam former 104. The data storage 107 stores the acoustic line signal output from the receive beam former 104 and the ultrasound image output from the ultrasound image generator 105. The control unit 108 controls each of the other components of the ultrasound diagnostic device 100.

Among the components of the ultrasound diagnostic device 100, the multiplexer 102, the transmission beam former 103, the receive beam former 104, and the ultrasound image generator 105 constitute ultrasound signal processing circuitry 150, and the ultrasound signal processing circuitry 150 constitutes an ultrasound signal processing device.

Each component of the ultrasound diagnostic device 100, for example, each of the multiplexer 102, the transmission beam former 103, the receive beam former 104, the ultrasound image generator 105, and the control unit 108 may be implemented by using a hardware circuit such as a field-programmable gate array (FPGA), an application-specific integrated circuit (ASIC), or the like. Alternatively, each of the components may be implemented by using a combination of software and a programmable device such as a processor. As a processor, a central processing unit (CPU) or a graphics processing unit (GPU) may be used for example, and a construction using a GPU is referred to as a General-purpose computing on graphics processing unit (GPGPU). Each of such components may be implemented as one circuit component, or as an aggregate of a plurality of circuit components. Further, a plurality of such components may be implemented by using one circuit component, or as an aggregate of a plurality of circuit components.

The data storage 107 is a computer-readable recording medium. For example, the data storage 107 may be implemented by using a flexible disk, a hard disk, an MO, a DVD, a DVD-RAM, a BD, or a semiconductor memory. Alternatively, the data storage 107 may be an external storage device connected to the ultrasound diagnostic device 100.

Note that the ultrasound diagnostic device 100 pertaining to the present embodiment need not have the structure illustrated in FIG. 1. For example, the ultrasound diagnostic device 100 may not include the multiplexer 102, and the transmission beam former 103 and the receive beam former 104 may be directly connected with each transducer element 101a of the probe 101. Further, the probe 101 may have built-in therein a part or the entirety of each of the transmission beam former 103, the receive beam former 104, and the like. Such modifications apply not only to the ultrasound diagnostic device 100 pertaining to the present embodiment, but also similarly apply to the ultrasound diagnostic devices described later in the other embodiments and modifications in the present disclosure.

### <Structure of Main Part of Ultrasound Diagnostic Device 100>

The ultrasound diagnostic device 100 pertaining to the embodiment is characterized for including the transmission beam former 103 and the receive beam former 104. The transmission beam former 103 causes the transducer elements 101a of the probe 101 to transmit ultrasound. The receive beam former 104 performs computation with respect to electric signals acquired through the reception of reflected ultrasound by the probe 101, and generates acoustic line signals used in forming an ultrasound image. Accordingly, the present disclosure focuses on the structure and the functions of each of the transmission beam former 103 and the receive beam former 104. Note that components other than the transmission beam former 103 and the receive beam former 104 may have structures and functions similar to those in conventional ultrasound diagnostic devices. In other words, the ultrasound diagnostic device 100 may be implemented by replacing beam formers in a conventional ultrasound diagnostic device with the beam formers pertaining to the present embodiment.

The following describes the structure of each of the transmission beam former 103 and the receive beam former 104.

### 1. Transmission Beam Former 103

The transmission beam former 103 is connected to the probe 101, via the multiplexer 102. However, note that the multiplexer 102 is not a mandatory element in the present invention. The transmission beam former 103 controls timings of application of high voltage with respect to each of a plurality of transducer elements 101a composing a transmission aperture Tx. The transmission aperture Tx is an array of transducer elements composed of all or some of the transducer elements 101a of the probe 101. Note that in the following, the term “transmission transducer element” is used to refer to transducer elements composing the transmission aperture Tx. The transmission beam former 103 includes a transmitter 1031.

The transmitter 1031 performs transmission processing. The transmission processing involves supplying a transmission signal having a pulsar waveform to each of the transmission transducer elements. A transmission transducer element receiving a transmission signal transmits an ultrasound beam. The transmitter 1031 supplies transmission signals to the transmission transducer elements based on transmission control signals output from the control unit 108. In specific, the transmitter 1031 includes, for example, a clock generation circuit, a pulse generation circuit, and a delay circuit. The clock generation circuit generates a clock signal specifying the transmission timing of ultrasound beams. The pulse generation circuit generates pulse signals for driving the transmission transducer elements. The delay circuit performs focus processing so that ultrasound beams are appropriately focused. In specific, the delay circuit sets a delay time for each transmission transducer element, and delays the transmission of the ultrasound beam from the transmission transducer element by the corresponding delay time.

The transmitter 1031 repetitively performs ultrasound transmission while shifting the transmission aperture Tx in the transducer element array direction each time, so that all of the transducer elements 101a of the probe 101 transmit ultrasound. In the present embodiment, a shift pitch Mp for each ultrasound transmission event corresponds to one transducer for convenience of explanation. However, the size of shift pitch Mp is not limited to this. Further, the transmitter 1031 outputs information indicating the positions of transmission transducer elements composing the transmission aperture Tx to the data storage 107, via the control unit 108. For example, supposing that the probe 101 has one hundred and ninety two (192) transducer elements 101a in total, the number of transmission transducer elements composing the transmission aperture Tx may be twenty (20) to one hundred (100). Further, in the present disclosure, the term transmission event is used to refer to ultrasound transmission by the transmitter 1031, performed by using one transmission aperture (i.e., one set of transmission transducer elements of the predetermined number).

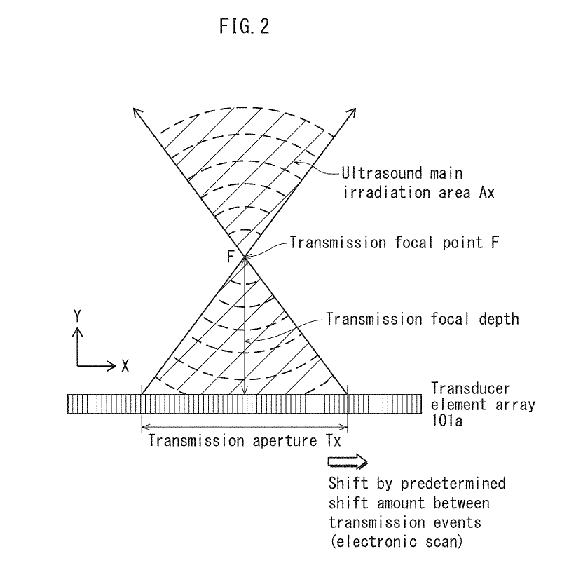

FIG. 2 is a schematic illustrating a propagation path of ultrasound transmitted by the transmission beam former 103. FIG. 2 illustrates a transmission aperture Tx for one transmission event (i.e., a transmission transducer element array composed of transmission transducer elements 101a that contribute to ultrasound transmission in the transmission event). Further, the transmission-array direction length of the transmission aperture Tx is considered the length of the transmission aperture Tx.

The transmission beam former 103 controls ultrasound transmission by the transmission transducer elements such that a transmission transducer element closer to the center position of the transmission aperture Tx transmits ultrasound later in the transmission event. Due to this, the wavefront of ultrasound transmitted from the transmission transducer elements composing the transmission aperture Tx converges at one point at a certain focal depth in the subject (i.e., the transmission focal point F). Note that the depth of the transmission focal point F (i.e., transmission focal depth) can be set as desired or required. After converging at the transmission focal point F, the wavefront of the transmitted ultrasound spreads out as before converging at the transmission focal point F. Thus, the transmitted ultrasound propagates through an hourglass-shaped area whose base is defined by the transmission aperture Tx and which is partitioned from other areas inside the subject by two straight lines intersecting at the transmission focal point F. More specifically, ultrasound transmitted from the transmission aperture Tx propagates in the following manner. As the transmitted ultrasound advances in a depth direction of the subject from the transmission aperture Tx, the width thereof (length along horizontal axis (X axis) in FIG. 2) gradually decreases until reaching the minimum width at the transmission focal point F. Then, as the transmitted ultrasound advances further in the depth direction from the transmission focal point F (i.e., as the ultrasound advances in the upward direction in FIG. 2), the width thereof increases (i.e., the ultrasound spreads out). In the following, the hourglass-shaped area described above is referred to as a ultrasound main irradiation area Ax. Note that as already described above, the transmission of ultrasound may be performed so that the ultrasound main irradiation area Ax converges at the focal area.

### 2. Receive Beam Former 104

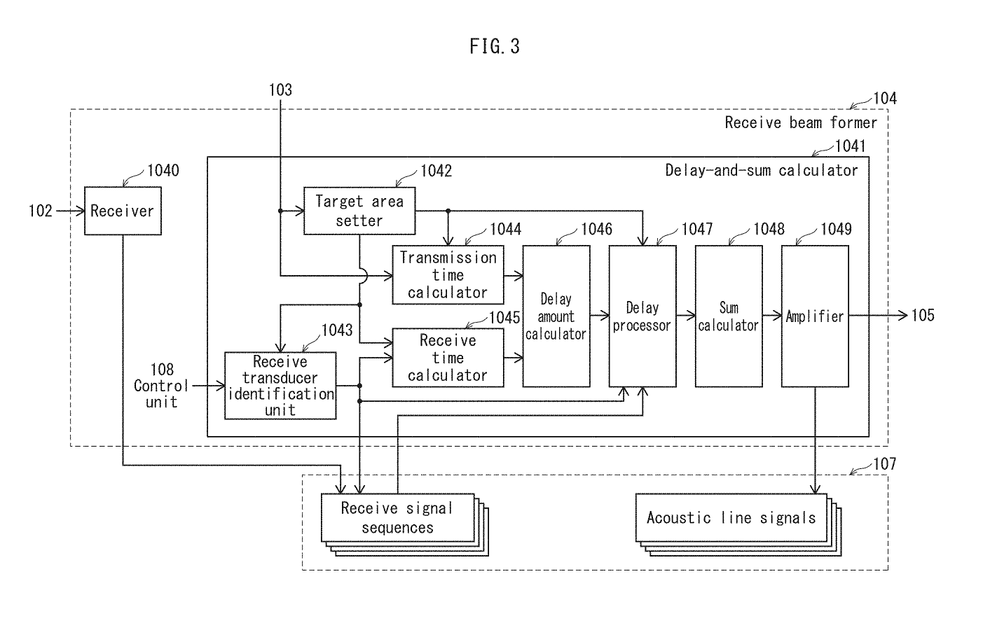

The receive beam former 104 generates acoustic line signals from electric signals acquired by a plurality of transducer elements 101a. The transducer elements 101a acquire the electric signals based on reflected ultrasound received by the probe 101. Here, an acoustic line signal for one measurement point is generated by performing delay-and-sum processing with respect to receive signals from the measurement point. Description of the delay-and-sum processing is provided later in the present disclosure. FIG. 3 is a functional block diagram illustrating the structure of the receive beam former 104. As illustrated in FIG. 3, the receive beam former 104 includes: a receiver 1040; and a delay-and-sum calculator 1041.

The following describes the structure of each functional block of the receive beam former 104.

### (1) Receiver 1040

The receiver 1040 is connected to the probe 101, via the multiplexer 102. However, note that the multiplexer 102 is not a mandatory element in the present invention. For each transmission event, the receiver 1040 generates receive signals (RF signals). The receiver 1040 generates the receive signals by first amplifying electric signals acquired through the probe 101 receiving reflected ultrasound, and then performing AID conversion on the amplified signals. The receiver 1040 performs the generation of receive signals for each transmission event, and outputs the receive signals to be stored in the data storage 107.

Here, the receiver 1040 generates one receive signal sequence (RF signal) for each of some or all of the transducer elements 101a of the probe 101. In specific, a receive signal sequence for a given transducer element is a digital signal yielded by performing A/D conversion on an electrical signal yielded through conversion of reflected ultrasound received by the transducer element, and is a sequence of signals along the ultrasound transmission direction (corresponding to the depth direction) that are received by the transducer element.

As discussed above, in each transmission event, the transmitter 1031 causes the plurality of transmission transducer elements composing the transmission aperture Tx, among the transducer elements 101a of the probe 101, each to transmit an ultrasound beam. Meanwhile, for each ultrasound transmission event, the receiver 1040, based on ultrasound reflection that each of some or all of the transducer elements composing the transmission apertures Tx acquires from the transmission event, generates a receive signal sequence for each of the transducer elements 101a having acquired the ultrasound reflection. In the present disclosure, each of the transducer elements 101a acquiring ultrasound reflection is referred to as “receive transducer element”. The number of receive transducer elements is equal to the number of transmission transducer elements composing the transmission aperture Tx.

Further, as already discussed above, the transmitter 1031 repetitively performs transmission events while shifting the transmission aperture Tx in the transducer element array direction each time, so that all of the transducer elements 101a of the probe 101 transmit ultrasound. Meanwhile, for each ultrasound transmission event, the receiver 1040 generates receive signal sequences for receive transducer elements 101a, and stores the receive signal sequences to the data storage 107.

### (2) Delay-and-Sum Calculator 1041

The delay-and-sum calculator 1041 sets a target area Bx for each transmission event. A target area Bx is an area in the subject in which reception beam forming is to be performed, and is composed of a plurality of measurement points Pij. Further, the delay-and-sum calculator 1041 identifies a receive transducer element Rij for each measurement point Pij of the target area Bx, and extracts a measurement point signal from a receive signal sequence received by the receive transducer element Rij. The delay-and-sum calculator 1041 performs delay-and-summing of measurement point signals for a plurality of transmission events based on position of the measurement point Pij to generate an acoustic line signal. As illustrated in FIG. 3, the delay-and-sum calculator 1041 includes: a target area setter 1042; a receive transducer identification unit 1043; a transmission time calculator 1044; a receive time calculator 1045; a delay amount calculator 1046; a delay processor 1047; a sum calculator 1048; and an amplifier 1049.

The following describes the structure of each functional block of the delay-and-sum calculator 1041.

### i) Target Area Setter 1042

The target area setter 1042 sets a target area Bx that is an area in the subject in which measurement point signals are to be identified. More specifically, in the present disclosure, the term “target area” is used to indicate a signal area for specifying a measurement point signal in the subject for one transmission event. Further, one measurement point signal is specified for each measurement point Pij of the target area Bx. In other words, the target area Bx is set for each transmission event in order to specify ones of the measurement points for which measurement point signals are to be specified for the transmission event.

Further, in the present disclosure, a measurement point signal is a group of receive signals that are generated from one transmission event. As already described above, from one transmission event, a plurality of measurement point signals are generated, each for a different one of the measurement points Pij of the target area Bx.

For each transmission event, the target area setter 1042 sets the target area Bx based on the information indicating the position of the transmission aperture Tx for the transmission event, which is acquired from the transmission beam former 103.

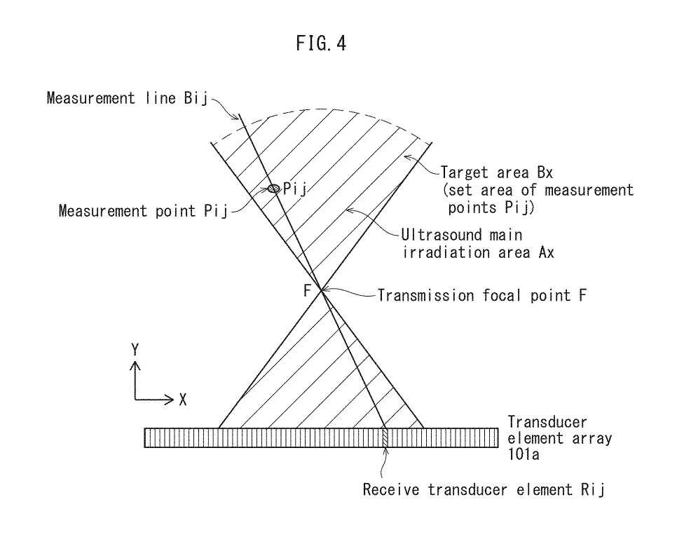

FIG. 4 is a schematic illustrating one example of the target area Bx. The target area Bx illustrated in FIG. 4 is equal to the entire ultrasound main irradiation area Ax. Note that the target area Bx is not limited by this case, and alternatively may be part of the ultrasound main irradiation area Ax.

The target area setter 1042 outputs the set target area Bx to the receive transducer identification unit 1043, the transmission time calculator 1044, the receive time calculator 1045, and the delay processor 1047.

ii) Receive transducer identification unit 1043

The receive transducer identification unit 1043 is a circuit that identifies a receive transducer element Rij for each measurement point Pij of the target area Bx based on the information indicating the position of the target area Bx from the target area setter 1042.

The receive transducer identification unit 1043 identifies, as a receive transducer element Rij for a measurement point Pij, a transducer element that is located on a straight line passing through the measurement point Pij and the transmission focal point. FIG. 4 is a schematic illustrating a relationship between the receive transducer element Rij identified by the receive transducer identification unit 1043 and the measurement point Pij. As illustrated in FIG. 4, based on an assumption that a measurement line Bij that is a straight line passes through the measurement point Pij and the transmission focal point F, a transducer element that is located on the measurement line Bij is identified as the receive transducer element Rij. Accordingly, a position of the receive transducer element Rij is determined by the position of the measurement point Pij and the transmission focal point F. That is, with respect to the measurement point Pij at the same position, the position of the receive transducer element Rij differs for each transmission event. Note that there is of course a possibility that a plurality of measurement points Pij at different positions and the transmission focal point F are located on a single straight line, that is, the measurement points Pij at the different positions are located on the same measurement line Bij. Thus, the number of receive transducer elements Rij corresponding to a measurement point Pij for each transmission event is of course one, but the number of measurement points Pij corresponding to a receive transducer element Rij for each transmission event is not limited to one, and there is a case where the receive transducer element Rij corresponds to a plurality of measurement points Pij for each transmission event. As described above, the target area Bx cannot include the outside of the ultrasound main irradiation area Ax, and thus the measurement points Pij inevitably exist inside the ultrasound main irradiation area Ax. As such, the measurement line Bij cannot pass through only the outside of the ultrasound main irradiation area Ax, and thus the receive transducer elements Rij are inevitably included in the transmission aperture Tx.

Identification of the receive transducer elements Rij is performed by the number of times equal to the number of measurement points Pij for each transmission event. Due to this, identification of the receive transducer elements Rij is repeated for the number of measurement points. Further, identification of the receive transducer elements Rij may be performed each time a transmission event is performed as described above, or alternatively, receive transducer elements Rij corresponding to respective measurement points Pij for multiple transmission events having been performed may be identified at once after the completion of the transmission events.

Further, in the above, description is provided that each receive transducer element Rij is a transducer element located on a measurement line Bij that is a straight line passing through the measurement point Pij and the transmission focal point F. Alternatively, in the case for example where a measurement line Bij passes through between two transducer elements, one of the two transducer elements may be identified as a receive transducer element Rij. Note that it is preferable that coordinates of the measurement point Pij should be set such that only one transducer element exists on the measurement line Bij. For example, the measurement point Pij should be set on only a straight line passing through any transducer element and the transmission focal point F.

Information indicating the receive transducer element Rij identified for each measurement point Pij is output to the receive time calculator 1045 and to the data storage 107 via the control unit 108.

The data storage 107 outputs a receive signal for each receive transducer element Rij to the delay processor 1047.

iii) Transmission Time Calculator 1044

The transmission time calculator 1044 is a circuit that, for each transmission event, calculates a transmission time for each measurement point Pij of the target area Bx for the transmission event. The transmission time for a given measurement point Pij is the time amount required for transmitted ultrasound to arrive at the measurement point Pij. The transmission time calculator 1044 acquires information indicating the positions of the transmission transducer elements for a given transmission event from the data storage 107, and information indicating the position of the target area Bx for the transmission event from the target area setter 1042. Based on such information, the transmission time calculator 1044, for each measurement point Pij located on the target areas composing the target area Bx, calculates the transmission time required for transmitted ultrasound to arrive at the measurement point Pij.

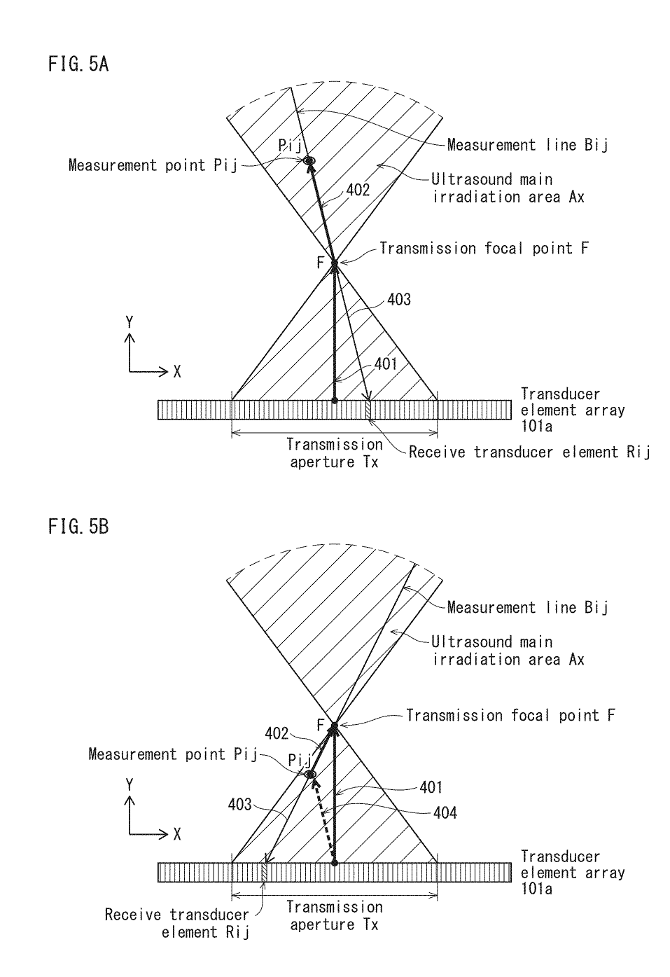

Each of FIGS. 5A and 5B is a schematic illustrating a propagation path of ultrasound that is transmitted from the transmission aperture Tx for a transmission event, is then reflected at a measurement point Pij of the target area Bx for the transmission event, and finally arrives at a receive transducer element Rij. Specifically, FIG. 5A illustrates the propagation path of ultrasound for a measurement point Pij located deeper than the transmission focal depth, whereas FIG. 5B illustrates the propagation path of ultrasound for a measurement point Pij located shallower than the transmission focal depth. Note that when comparing the position of a measurement point Pij located deeper than the transmission focal depth and the position of a measurement point Pij located shallower than the transmission focal depth, the measurement point Pij located deeper than the transmission focal depth is located relatively far from the probe and the measurement point Pij located shallower than the transmission focal depth is located relatively near to the probe.

Following emission of ultrasound from the transmission aperture Tx, the wavefront of ultrasound converges at the transmission focal point F after proceeding along the path 401. Subsequently, the wavefront spreads out once again and arrives at the measurement point Pij. When there is a change in acoustic impedance at the measurement point Pij, transmitted ultrasound generates ultrasound reflection, which is received by the receive transducer elements Rij. The transmission focal point F is preset in advance upon designing of the transmission beam former 103. Thus, the length of the path 402 from the transmission focal point F to the measurement point Pij can be calculated geometrically.

The following describes how the transmission time is calculated in further detail.

First, the calculation of a transmission time for a measurement point Pij located deeper than the transmission focal depth is described, with reference to FIG. 5A. A transmission time for a measurement point Pij located deeper than the transmission focal depth is calculated assuming that ultrasound transmitted from the transmission aperture Tx arrives at the transmission focal point F by traveling along path 401, and then arrives at the measurement point Pij by traveling along path 402 from the transmission focal point F. As such, the transmission time for such a measurement point Pij is the total of the time amount required for transmitted ultrasound to travel through path 401 and the time amount required for transmitted ultrasound to travel through path 402. Specifically, the transmission time for such a measurement point Pij can be calculated, for example, by dividing the total of the lengths of paths 401 and 402 by the velocity at which ultrasound propagates within the subject.

In the meantime, the following describes the calculation of a transmission time for a measurement point Pij located shallower than the transmission focal depth, with reference to FIG. 5B. A transmission time for a measurement point Pij located shallower than the transmission focal depth is calculated assuming that the time amount required for ultrasound transmitted from the transmission aperture Tx to arrive at the transmission focal point F by travelling along path 401 equals the time amount required for ultrasound transmitted from the transmission aperture Tx to travel along path 404 to arrive at the measurement point Pij and then travel along path 402 to arrive at the transmission focal point F from the measurement point Pij. As such, the transmission time for such a measurement point Pij is calculated by subtracting the time amount required for transmitted ultrasound to travel through the path 402 from the time amount required for transmitted ultrasound to travel through the path 401. Specifically, a transmission time for such a measurement point Pij can be calculated, for example, by dividing the value acquired by subtracting the length of path 402 from the length of path 401, by the velocity at which ultrasound propagates within the subject.

Note that in the present embodiment, a transmission time for a measurement point Pij located at the transmission focal depth is calculated in the same way as the transmission time for a measurement point Pij located deeper than the transmission focal depth. That is, a transmission time for a measurement point Pij located at the transmission focal depth is calculated by using the total of the time amount required for transmitted ultrasound to travel through path 401 and the time amount required for transmitted ultrasound to travel through path 402. Alternatively, a transmission time for a measurement point Pij located at the transmission focal depth may be calculated in the same way as the transmission time for a measurement point Pij located shallower than the transmission focal depth, or that is by using a value obtained by subtracting the time amount required for transmitted ultrasound to travel through the path 402 from the time amount required for transmitted ultrasound to travel through the path 401. This is because the length of the path 402 is zero in this case, and thus, the transmission time for a measurement point Pij located at the transmission focal depth equals the time amount required for transmitted ultrasound to travel through path 401 with either calculation method.

For each transmission event, the transmission time calculator 1044 calculates the transmission time for each measurement point Pij of the target area Bx for the transmission event. That is, the transmission time calculator 1044 calculates, for each measurement point Pij, the time amount required for transmitted ultrasound to arrive at the measurement point Pij. Further, the transmission time calculator 1044 outputs the transmission time so calculated to the delay amount calculator 1046.

### iv) Receive Time Calculator 1045

The receive time calculator 1045 is a circuit that calculates, for each measurement point Pij, a receive time required for ultrasound reflection from the measurement point Pij to arrive at a corresponding receive transducer element Rij. For a given transmission event, the receive time calculator 1045 acquires information indicating the positions of the receive transducer elements Rij for the given transmission event from the receive transducer identification unit 1043. Based on such information, the receive time calculator 1045, for each measurement point Pij of the target area Bx, calculates the receive time required for transmitted ultrasound to arrive at a corresponding receive transducer element Rij after being reflected at the measurement point Pij.

As already discussed above, transmitted ultrasound arriving at a measurement point Pij generates ultrasound reflection when there is a change in acoustic impedance at the measurement point Pij. The reflected ultrasound is then received by receive transducer elements Rij of the probe 101. As discussed above, the receive time calculator 1045 acquires information indicating the positions of the receive transducer elements Rij corresponding to the respective measurement points Pij from the receive transducer identification unit 1043. Accordingly, the receive time calculator 1045 is able to geometrically calculate the length of paths 403 leading from the measurement points Pij to the respective receive transducer elements Rij.

For each transmission event, the receive time calculator 1045 calculates the receive time for each measurement point Pij of the target area Bx for the transmission event. That is, the receive time calculator 1045 calculates, for each measurement point Pij, the time required for transmitted ultrasound to arrive at a corresponding receive transducer element Rij after being reflected at the measurement point Pij. Further, the receive time calculator 1045 outputs the receive time so calculated to the delay amount calculator 1046.

### v) Delay Amount Calculator 1046

The delay amount calculator 1046 is a circuit that calculates, for each receive transducer element Rij, a total propagation time based on the transmission time and the receive time for the receive transducer element Rij. Further, the delay amount calculator 1046 calculates, for each receive transducer element Rij, a delay amount to be applied to a receive signal sequence for the receive transducer element Rij. In specific, the delay amount calculator 1046 acquires, from the transmission time calculator 1044, the transmission time required for ultrasound waves to arrive at a measurement point Pij. Further, for each receive transducer element Rij, the delay amount calculator 1046 acquires, from the receive time calculator 1045, the receive time required for ultrasound to be reflected at the measurement point Pij and arrive at the receive transducer element Rij. Then, the delay amount calculator 1046, for each receive transducer element Rij, calculates a total propagation time required for transmitted ultrasound to arrive at the receive transducer element Rij after being reflected at the measurement point Pij. Further, the delay amount calculator 1046 calculates a delay amount for each receive transducer element Rij. For each measurement point Pij of the target area Bx, the delay amount calculator 1046 calculates, for each receive transducer element Rij, the delay amount to be applied to a receive signal sequence for the receive transducer element Rij, and outputs the delay amounts to the delay processor 1047.

### vi) Delay Processor 1047

The delay processor 1047 is a circuit that specifies, for each receive transducer element Rij, a receive signal based on reflected ultrasound from a measurement point Pij. In specific, for each receive transducer element Rij, the delay processor 1047 specifies a receive signal corresponding to the delay amount for the receive transducer element Rij from the receive signal sequence for the receive transducer element Rij.

More specifically, for each transmission event, the delay processor 1047 acquires, for each receive transducer element Rij, information indicating the position of the receive transducer element Rij from the receive transducer identification unit 1043, the receive signal sequence for the receive transducer element Rij from the data storage 107, and the delay amount to be applied to the receive signal sequence of the receive transducer element Rij from the delay amount calculator 1046. Further, for each receive transducer element Rij, the delay processor 1047 specifies a measurement point signal based on reflected ultrasound from a measurement point Pij. In specific, the delay processor 1047 specifies as a measurement point signal, from the receive signal sequence for the receive transducer element Rij, a receive signal corresponding to a time point after subtraction of the delay amount for the receive transducer element Rij. The delay processor 1047 outputs the measurement point signal so specified to the sum calculator 1048.

vii) Sum Calculator 1048

The sum calculator 1048 is a circuit that generates a delayed-and-summed acoustic line signal for each measurement point Pij, by using as input the specified measurement point signal for the measurement point Pij, which is output from the delay processor 1047, and performing weighted summing of the measurement point signals for multiple transmission events based on the position of the measurement point Pij. The sum calculator 1048 sums the receive signals for the receive transducer elements Rij corresponding to the measurement point Pij, after the receive signals have been put in the same phase by the delay processor 1047. Due to this, the sum calculator 1048 is capable of overlaying the measurement point signals based on reflected ultrasound from the measurement point Pij for transmission events, and thereby increasing S/N ratio of the measurement point signals to generate an acoustic line signal for the measurement point Pij. Note that all weighting coefficients for weighted summing may be one, or alternatively may be arbitrarily set.

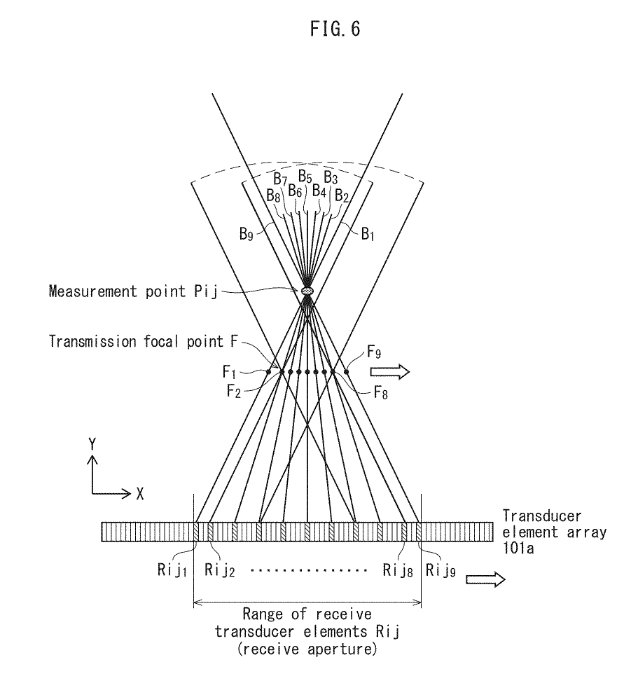

FIG. 6 is a schematic illustrating processing by the adder 1048 for generating an acoustic line signal. As already discussed above, ultrasound transmission is performed by repetitively performing transmission events while shifting the transmission transducer element array (i.e., the transmission aperture Tx) by a width of a single transducer element in the transducer element array direction each time. Due to this, the transmission focal points F for two consecutive transmission events differ in position from one another in the transducer element array direction by the width of a single transducer element, and thus the receive transducer element Rij shifts in synchronization with shift of the transmission focal point F. In specific, a receive transducer element Rij₁ on a measurement line B₁ is identified for a transmission event for a transmission focal point F₁, a receive transducer element Rij₂ on a measurement line B₂ is identified for a transmission event for a transmission focal point F₂, . . . , a receive transducer element Rij₈ on a measurement line B₈ is identified for a transmission event for a transmission focal point F₈, and a receive transducer element Rij₉ on a measurement line B₉ is identified for a transmission event for a transmission focal point F₉. Further, the sum calculator 1048 generates an acoustic line signal by synthesizing the measurement point signals corresponding to the receive transducer element Rij for transmission events based on the position of the measurement point Pij. Accordingly, a positional relationship between the transmission focal point F and the measurement point Pij and a positional relationship between the measurement point Pij and the receive transducer element Rij differ between transmission events, and thus diversity thereof is greatly ensured. In specific, the transmission focal point F is set so as to be included in a range from the transmission focal points F₁ to F₉. The transmission focal point F₁ corresponds to the measurement point Pij on a right edge of the ultrasound main irradiation area Ax, and the transmission focal point F₉ corresponds to the measurement point Pij on a left edge of the ultrasound main irradiation area Ax. The receive transducer element Rij is set so as to be included in a range from the receive transducer elements Rij₁ to Rij₉ in synchronization with the transmission focal point. This range from the receive transducer elements Rij₁ to Rij₉ compose a receive aperture. Due to this, it is possible to maximize the effect of improving spatial resolution and S/N ratio by virtual transmission beam forming.

Further, for a measurement point Pij included in multiple target areas Bx, values of a plurality of measurement point signals are summed. Thus, the acoustic line signal for such a measurement point may indicate a great value, depending upon the number of target areas Bx in which the measurement point is included. In the following, the number of different target areas Bx in which a given measurement point is included is referred to as an overlap count of the measurement point, and the maximum value of the overlap count in the transducer element array direction is referred to as a maximum overlap count.

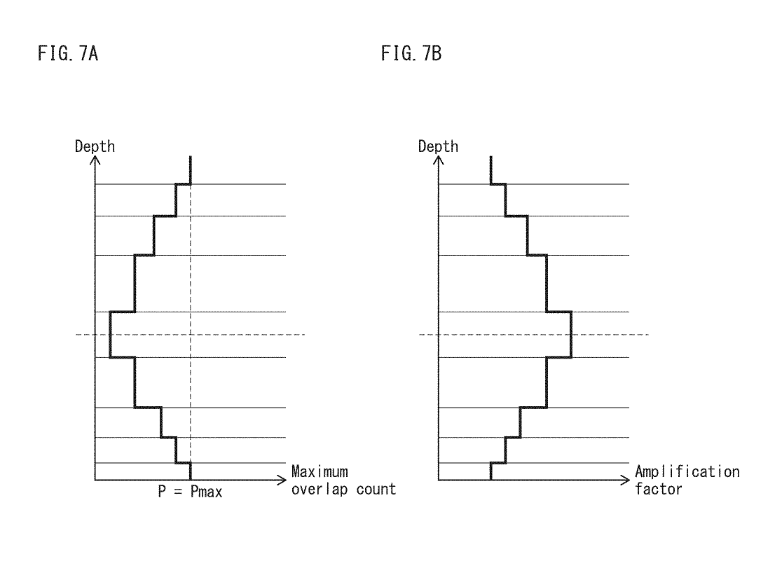

Further, in the present embodiment, the target area Bx has an hourglass-shape. Due to this, the overlap count and the maximum overlap count fluctuate in the depth direction of the subject, as illustrated in FIG. 7A. Accordingly, there is a depth-direction fluctuation in values of synthesized acoustic line signals.

As a result of one transmission event and processing accompanying the transmission event, a measurement point signal is specified for each measurement point Pij of the target area Bx for the transmission event. Further, by repetitively performing transmission events while shifting the transmission aperture Tx in the transducer element array direction each time, all of the transducer elements 101a in the probe 101 perform ultrasound transmission, and the sum calculator 1048 overlays measurement point signals acquired by the ultrasound transmission to generate an acoustic line signal for each measurement point Pij within one frame.

The acoustic line signal generated by the adder 1048 is output to the amplifier 1049.

viii) Amplifier 1049

As already described above, there is a depth-direction fluctuation in values of acoustic line signals. In order to moderate such fluctuation in values of different acoustic line signals, the amplifier 1049, in generating acoustic line signals, performs amplification of multiplying the acoustic line signals by amplification factors. Here, the amplifier 1049 determines an amplification factor for a given acoustic line signal according to the number of measurement point signals synthesized to yield the acoustic line signal.

FIG. 7B is a schematic providing an overview of the amplification performed by the amplifier 1049. The maximum overlap count fluctuates in the depth direction, as illustrated in FIG. 7B. Thus, to compensate with this fluctuation in maximum overlap count, the amplifier 1049 multiplies the acoustic line signals by respective amplification factors that are based on the maximum overlap counts and vary in the depth direction. This moderates a difference between values of acoustic line signals deriving from the fluctuation in overlap counts in the depth direction, and thus, the values of the acoustic line signals after the amplification are averaged out in the depth direction. That is, the amplification performed by the amplifier 1049 is gain equalization in the depth direction.

Further, the amplifier 1049 may also multiply the synthesized acoustic line signals by amplification factors varying in the transducer element array direction that are calculated based on overlap counts, when overlap counts fluctuate in the transducer element array direction. This moderates a difference between values of synthesized acoustic line signals deriving from the fluctuation in overlap counts in the transducer element array direction, and thus, the values of the synthesized acoustic line signals after the amplification are averaged out in the transducer element array direction.

Here, note that the amplifier 1049 may generate the acoustic line signal by synthesizing amplified synthesized acoustic line signals for respective measurement points.

### <Operations>

The following describes the operations of the ultrasound diagnostic device 100 having the structure described up to this point.

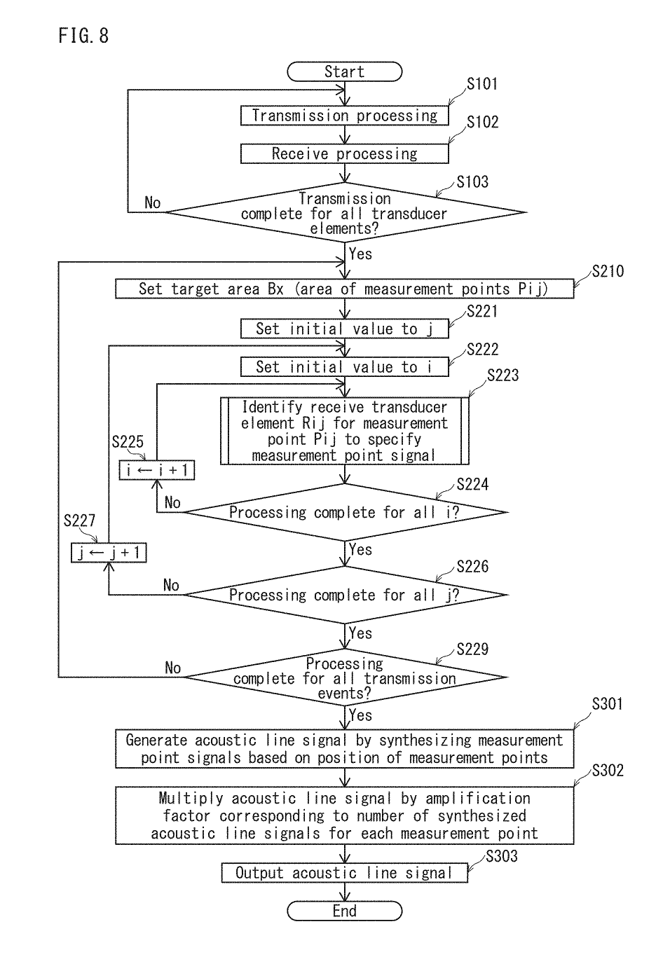

FIG. 8 is a flowchart illustrating beam forming by the receive beam former 104.

First, in Step S101, the transmitter 1031 performs transmission processing (a transmission event) of supplying a transmission signal causing transmission of an ultrasound beam to each transmission transducer element of the transmission aperture Tx.

In Step S102, the receiver 1040 generates receive signal sequences based on electric signals yielded through the reception of reflected ultrasound by the probe 101, and outputs the receive signal sequences to be stored in the data storage 107. Then, a determination is made of whether or not all transducer elements 101a of the probe 101 have performed ultrasound transmission (S103). When one or more of the transducer elements 101a have not yet performed ultrasound transmission, processing returns to Step S101, which results in another transmission event being executed by shifting the transmission aperture Tx in the transducer element array direction by the width of a single transducer element. Meanwhile, when all of the transducer elements 101a have performed ultrasound transmission, processing proceeds to Step S210.

In Step S210, the target area setter 1042 sets a target area Bx for a processing-target transmission event based on information indicating the position of the transmission aperture Tx for the processing-target transmission event. In the initial loop of processing, the target area setter 1042 sets a target area Bx for the initial transmission event, which can be calculated from the transmission aperture Tx for the initial transmission event.

Subsequently, processing proceeds to reception beam forming (Steps S221 through S227). First, coordinate values i and j indicating a position of a measurement point Pij of the target area Bx for the processing-target transmission event are initialized (set to the respective minimum possible values in the target area Bx) (Steps S221 and S222).

Subsequently, a measurement point signal is specified for the current measurement point Pij (Step S223).

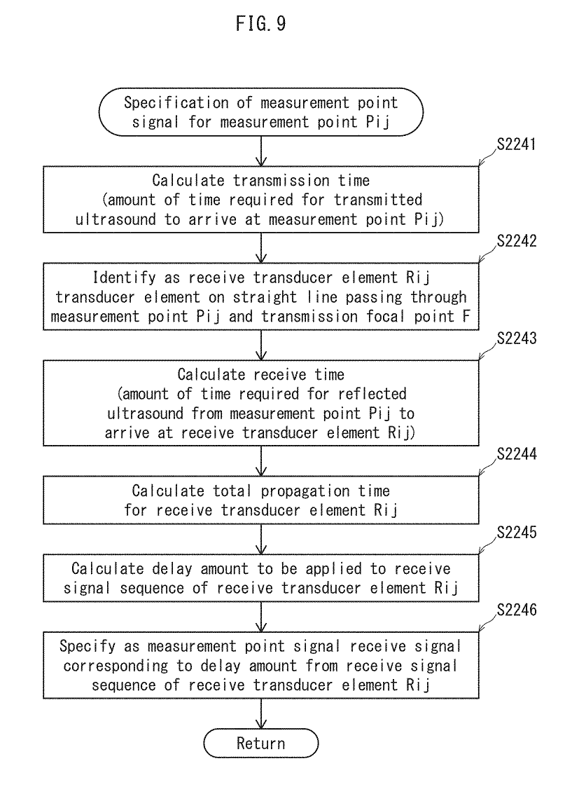

The following describes the operations in Step S223 for specifying a measurement point signal for the current measurement point Pij. FIG. 9 is a flowchart illustrating the operations of the receive beam former 104 for specifying the measurement point signal for the current measurement point Pij.

First, in Step S2241, the transmission time calculator 1044 calculates, for the current measurement point Pij, a transmission time required for transmitted ultrasound to arrive at the current measurement point Pij. As already described above, the current measurement point Pij is a measurement point of the target area Bx for the processing-target transmission event. Here, (i) when the current measurement point Pij is located at the transmission focal depth or deeper than the transmission focal depth, the transmission time for the current measurement point Pij is calculated by dividing, by ultrasound velocity cs, the geometrically-calculable length of a path (combination of paths 401 and 402) starting at a transmission transducer element in the transmission aperture Tx and reaching the current measurement point Pij via the transmission focal point F. Meanwhile, (ii) when the current measurement point Pij is located shallower than the transmission focal depth, the transmission time for the current measurement point is calculated by dividing, by the ultrasound velocity cs, a value (401−402) obtained by subtracting the geometrically-calculable length of the path from the transmission focal point F to the current measurement point Pij from the geometrically-calculable length of the path from a transmission transducer element in the transmission aperture Tx to the transmission focal point F.

Subsequently, in Step S2242, the receive transducer identification unit 1043 identifies, as the receive transducer element Rij, a transducer that is located on a straight line passing through the measurement point Pij and the transmission focal point F.

Then, the receive time for the target receive transducer element Rij is calculated (Step S2243). The receive time is the time required for transmitted ultrasound to arrive at the target receive transducer element Rij after being reflected at the current measurement point Pij. The receive time for the target receive transducer element Rij can be calculated by dividing, by the ultrasound velocity cs, the geometrically-calculable length of the path 403 from the current measurement point Pij to the target receive transducer element Rij. Further, from a sum of the transmission time and the receive time for the target receive transducer element Rij, the total propagation time required for ultrasound transmitted from the transmission aperture Tx to arrive at the target receive transducer element Rij after being reflected at the current measurement point Pij is calculated (Step S2244). Further, based on the total propagation time for the receive transducer element Rij, the delay amount for the target receive transducer element Rij is calculated (Step S2245).

In Step S2246, the delay processor 1047, for each receive transducer element Rij, specifies a measurement point signal based on reflected ultrasound from the current measurement point Pij. Here, the delay processor 1047 specifies, from a receive signal sequence corresponding to each receive transducer element Rij, a receive signal corresponding to a time point after subtraction of the delay amount for the receive transducer element Rij.

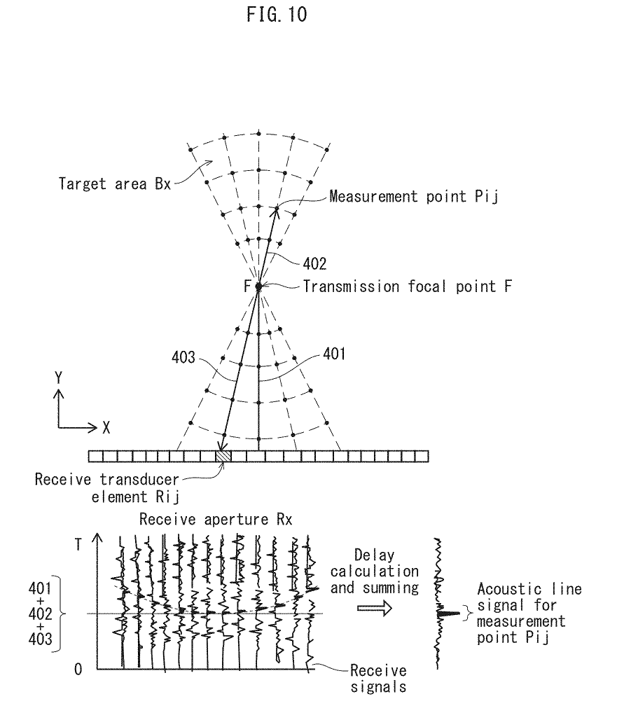

Referring to FIG. 8 once again, subsequently, a measurement point signal is specified for each measurement point Pij (each illustrated in FIG. 10 as a black dot) of the target area Bx for the processing-target transmission event, by repeating Step S223 while incrementing the coordinate values i and j. Subsequently, a determination is performed of whether or not a measurement point signal has been specified for every measurement point Pij of the target area Bx (Steps S224 and S226). When a measurement point signal has not yet been specified for every measurement point Pij of the target area Bx, the coordinate values i and j are incremented (Steps S225 and S227), yielding a measurement point signal for another measurement point Pij (Step S222). Meanwhile, when a measurement point signal has already been specified for every measurement point Pij of the target area Bx, processing proceeds to Step S229. At this point, a measurement point signal has already been specified for each measurement point Pij of the target area Bx corresponding to the processing-target transmission event.

Subsequently, a determination is performed of whether or not a measurement point signal has been specified for each transmission event having been performed (Step S229). When measurement point signals have not yet been specified for one or more transmission events, processing proceeds to Step S210, where the coordinate values i and j are initialized (set to the respective minimum possible values in the target area Bx for the subsequent transmission event, which can be calculated from the transmission aperture Tx for the subsequent transmission event) (Steps S221 and S222), and then a measurement point signal is specified (Step S223). Meanwhile, when measurement point signals have been specified for every transmission event having been performed, processing proceeds to Step S301.

In Step S301, the adder 1048 synthesizes the measurement point signals based on the position of each measurement point Pij. Thus, an acoustic line signal is generated for each measurement point Pij. Subsequently, the amplifier 1049 multiplies each acoustic line signal by a corresponding amplification factor that is determined based on the number of measurement point signals that have been synthesized to yield the acoustic line signal (Step S302). Further, the amplifier 1049 outputs the amplified acoustic line signal to the ultrasound image generator 105 and the data storage 107 (Step S303), and processing is terminated.

### <Conclusion>

As described above, the ultrasound diagnostic device 100 pertaining to the present embodiment, according to the synthetic aperture method, generates an acoustic line signal by performing delay-and-summing of measurement point signals for the same measurement point Pij that are specified from different transmission events. This achieves the effect of performing, for multiple transmission events, virtual transmission focusing even for measurement points that are located in depths other than that of the transmission focal point F. This improves spatial resolution and S/N ratio.

Also, the ultrasound diagnostic device 100 performs delay-and-summing for a plurality of transmission events. This supplements receive signals based on ultrasound reflection from the measurement point Pij while putting the receive signals in the same phase. Accordingly, it is possible to improve signal S/N ratio even by performing drastic reduction of computation amount according to which no sum-and-delaying for each transmission event is performed.

Further, according to conventional ultrasound diagnostic devices, weighting is performed such that a higher weight sequence is applied to a transducer element with a smaller distance from the measurement point Pij. Compared to this, the ultrasound diagnostic device 100 does not perform weighting in delay-and-summing. In the conventional ultrasound diagnostic devices, since delay-and-summing is performed for each transmission event, reception apodization is performed such that the greatest weight is added to a transducer element with the smallest distance from the measurement point Pij. In the ultrasound diagnostic device 100 compared with this, as a distance increases between the measurement point Pij and the receive transducer element Rij, a distance inevitably increases between the measurement point Pij and the transmission focal point F. That is, as the distance increases between the measurement point Pij and the receive transducer element Rij, decay of ultrasound arriving at the measurement point Pij increases. In other words, as the distance increases between the measurement point Pij and the receive transducer element Rij, ultrasound reflected from the measurement point Pij decreases. Accordingly, in the ultrasound diagnostic device 100, since measurement point signals are in the state that is the same as the state in which reception apodization has been performed, further weighting is not necessary.

Further, the ultrasound diagnostic device 100 sets the entire ultrasound main irradiation area Ax as the target area in which measurement points are to be set. This increases diversity of an ultrasound beam transmission path from the transmission aperture Tx to the measurement point Pij and a reflected ultrasound receive path from the measurement point Pij to the receive transducer element Rij, thereby exhibiting the maximum effect of suppressing a decrease in spatial resolution and signal S/N ratio by virtual transmission beam forming. Further, since the receive transducer element Rij differs for each transmission event, sensitivity difference is unlikely to occur between the measurement points Pij even with variation in sensitivity of transducer elements, thereby suppressing influence on ultrasound images.

Accordingly, the present embodiment considerably reduces computation amount compared to ultrasound diagnostic devices using conventional synthetic aperture methods while suppressing degradation of acoustic line signal quality, and contributes to a reduction of processor cost.

### <<Modification 1>>

In embodiment 1, description is provided of the case where a receive transducer element Rij with respect to each measurement point Pij is identified for each transmission event, a measurement point signal is specified based on a reflected ultrasound from the measurement point Pij received by the receive transducer element Rij, and delay-and-summing of measurement point signals is performed for a plurality of transmission events, thereby generating an acoustic line signal.

In modification 1, in addition to the receive transducer element Rij, another receive transducer element is further identified with respect to each measurement point Pij for each transmission event, two measurement point signals are specified based on reflected ultrasounds from the measurement point Pij. Then, delay-and-summing of measurement point signals, each two of which are specified for one transmission event, is performed for a plurality of transmission events, thereby generating an acoustic line signal. This is a difference from embodiment 1. Modification 1 has operations and structures that are the same as those in embodiment 1 except for operations and structures for identification of receive transducer elements, specification of measurement point signals, and delay-and-summing. Thus, description for the same parts is omitted.

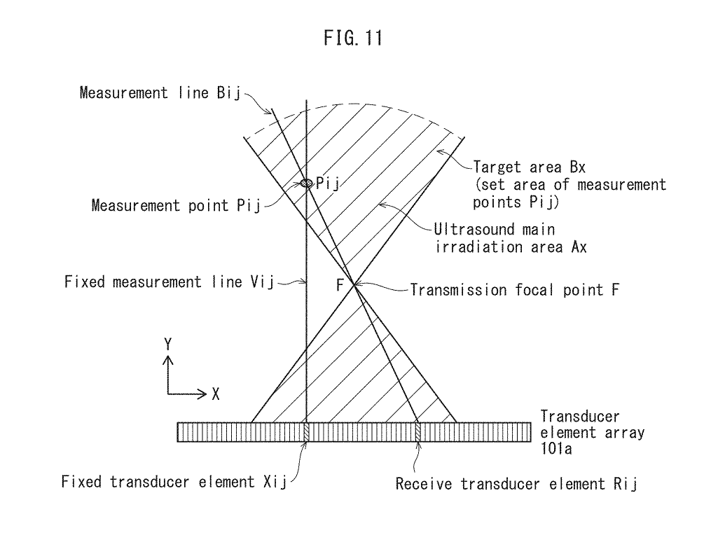

FIG. 11 is a schematic illustrating a relationship between receive transducer elements identified by the receive transducer identification unit and measurement points. A receive transducer element Rij is a transducer element that is located on a measurement line Bij that is a straight line passing through a measurement point Pij and the transmission focal point F. The details thereof are described in embodiment 1, and thus its description is omitted here. Meanwhile, in modification 1, a fixed receive transducer element Xij is identified in addition to the receive transducer element Rij. The fixed receive transducer element Xij is identified as illustrated in FIG. 11. In specific, assume a fixed measurement line Vij that is a straight line that passes through a measurement point Pij and is perpendicular to a direction in which the transducer elements of the transducer elements 101a are arrayed (the X direction) (that is, a straight line extending in the Y direction). An element that is located on the fixed measurement line Vij is identified as the fixed receive transducer element Xij. In other words, the fixed measurement line Vij is a transducer element that is closest to the measurement point Pij. The position of the fixed receive transducer element Xij is determined uniquely based on the position of the measurement point Pij irrespective of the position of the transmission focal point F. That is, the fixed receive transducer element Xij for a measurement point Pij that is located on the same position is constant for a plurality of transmission events. Note that one fixed receive transducer element Xij may correspond to a plurality of measurement points Pij, like the receive transducer element Rij.

Identification of the fixed receive transducer element Xij is performed by the number of times equal to the number of the measurement points Pij. Identification of the fixed receive transducer element Xij may be performed simultaneously with identification of the receive transducer element Rij, or alternatively may be performed separately from identification of the receive transducer element Rij. For example, fixed receive transducer elements Xij corresponding to measurement points Pij for a plurality of transmission events are identified after completion of the transmission events.

Information indicating the fixed receive transducer element Xij identified for each measurement point Pij is output to the receive time calculator and to the data storage unit via the control unit, like the information indicating the receive transducer element Rij.

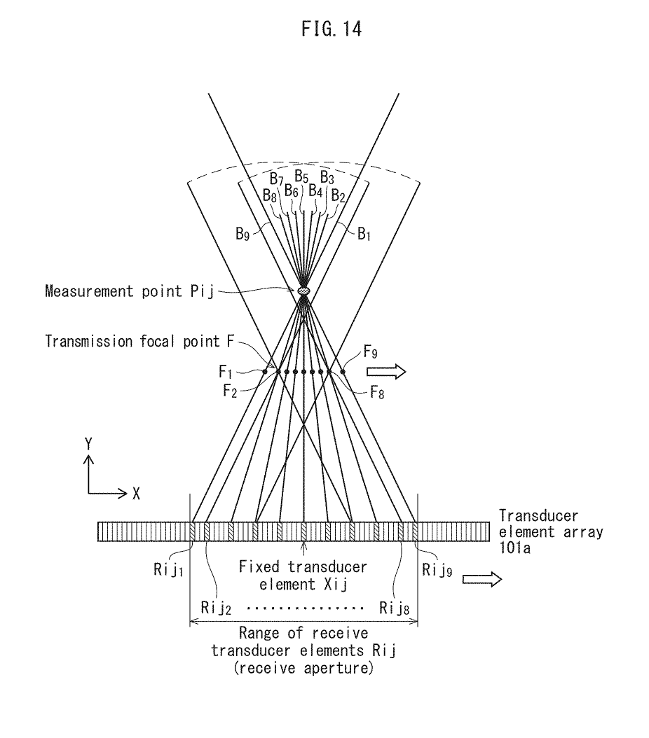

FIG. 14 is a schematic illustrating processing for generating an acoustic line signal. As already discussed above, ultrasound transmission is performed by repetitively performing transmission events while shifting the transmission transducer element array (i.e., the transmission aperture Tx) by a width of a single transducer element in the transducer element array direction each time. Due to this, the transmission focal points F for two consecutive transmission events differ in position from one another in the transducer element array direction by the width of a single transducer element, and thus the receive transducer element Rij shifts in synchronization with shift of the transmission focal point F. Meanwhile, the position of the fixed receive transducer element Xij is constant for a plurality of transmission events. Further, an acoustic line signal is generated by synthesizing measurement point signals corresponding to the receive transducer element Rij and the fixed receive transducer element Xij for transmission events based on the position of the measurement point Pij. Accordingly, a positional relationship between the transmission focal point F and the measurement point Pij and a positional relationship between the measurement point Pij and the receive transducer element Rij differ between transmission events, and thus diversity thereof is greatly ensured. The range from the receive transducer elements Rij₁ to Rij₉ compose a receive aperture, like in embodiment 1. Due to this, it is possible to maximize the effect of improving spatial resolution and S/N ratio by virtual transmission beam forming.

### <Operations>

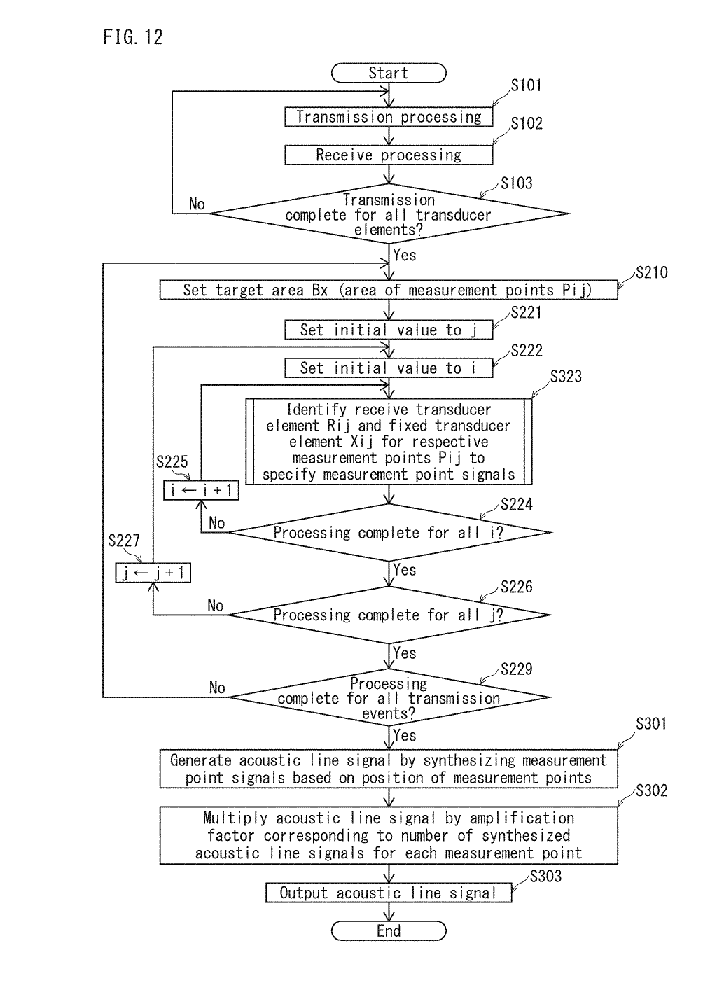

FIG. 12 is a flowchart illustrating beam forming by a receive beam former pertaining to modification 1. The flowchart in FIG. 12 differs from the flowchart in FIG. 8 for identification of a receive transducer element Rij and a fixed receive transducer element Xij corresponding to respective measurement points Pij for specification of two measurement point signals (Step S323) being performed in place of identification of the receive transducer element Rij corresponding to the measurement point Pij for specification of the measurement point signal (Step S223). Meanwhile, the processing in steps other than Step S323 in the flowchart in FIG. 12 is similar to the processing in the corresponding steps in the flowchart in FIG. 8. Thus, description of such similar processing is not provided in the following.

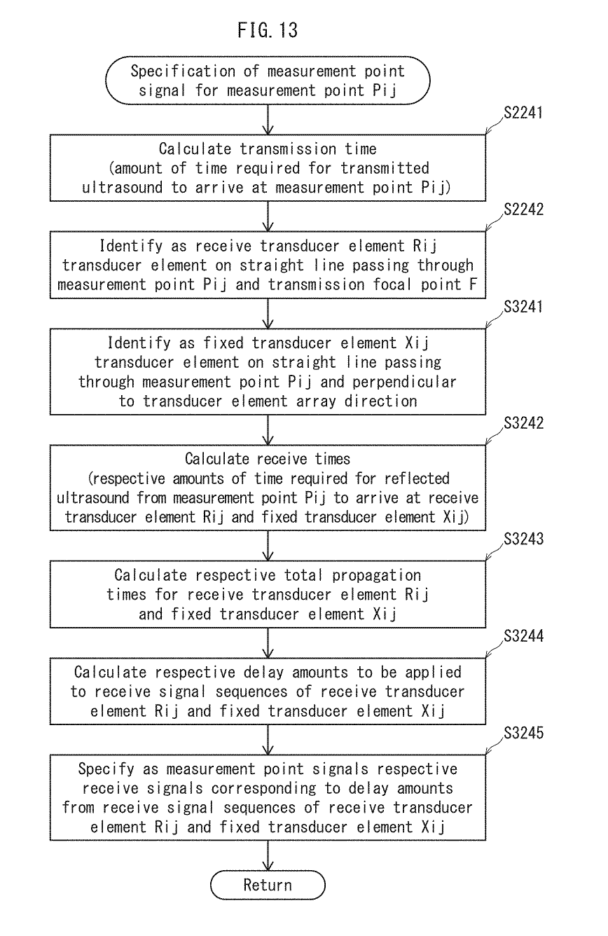

The following describes in detail specification of the measurement point signals for the measurement point Pij (Step S323). FIG. 13 is a flowchart illustrating specification of measurement point signals for the measurement point Pij by the receive beam former. Note that steps in FIG. 13 that are the same as those in FIG. 9 have the same step numbers, and detailed description thereof is omitted.

First, in Step S2241, the transmission time calculator calculates, for the current measurement point Pij, a transmission time required for transmitted ultrasound to arrive at the current measurement point Pij. As already described above, the current measurement point Pij is a measurement point of the target area Bx for the processing-target transmission event.

Subsequently, in Step S2242, the receive transducer identification unit identifies, as a receive transducer element Rij, a transducer element that is located on a straight line passing through the measurement point Pij and the transmission focal point F.

Subsequently, in Step S3241, the receive transducer identification unit identifies, as a fixed receive transducer element Xij, a transducer element that is located on a straight line passing through the measurement point Pij and is perpendicular to the transducer element array direction.

Subsequently, the receive transducer identification unit calculates respective receive times for the target receive transducer element Rij and the fixed receive transducer element Xij (Step S3242). The respective receive times for the target receive transducer element Rij and the fixed receive transducer element Xij are the times required for transmitted ultrasound to arrive at the target receive transducer element Rij and the fixed receive transducer element Xij and after being reflected at the current measurement point Pij in the subject body. The respective receive times for the target receive transducer element Rij and the fixed receive transducer element Xij can be calculated by dividing, by the ultrasound velocity cs, the geometrically-calculable length of the path from the current measurement point Pij to the target receive transducer element Rij and the geometrically-calculable length of the path from the current measurement point Pij to the fixed receive transducer element Xij, respectively. Further, from a sum of the transmission time and the receive time for the target receive transducer element Rij, the total propagation time is calculated which is required for ultrasound transmitted from the transmission aperture Tx to arrive at the target receive transducer element Rij after being reflected at the current measurement point Pij. Similarly, from a sum of the transmission time and the receive time for the fixed receive transducer element Xij, the total propagation time is required which is required for ultrasound transmitted from the transmission aperture Tx to arrive at the fixed receive transducer element Xij after being reflected at the current measurement point Pij (Step S3243). Further, the delay amount for the target receive transducer element Rij is calculated based on the total propagation time for the target receive transducer element Rij, and the delay amount for the fixed receive transducer element Xij is calculated based on the total propagation time for the fixed receive transducer element Xij (Step S3244).

Subsequently, in Step S3245, the delay processor specifies measurement point signals based on reflected ultrasound from the current measurement point Pij. In specific, the delay processor specifies a receive signal corresponding to a time point after subtraction of the delay amount for the receive transducer element Rij from a receive signal sequence corresponding to the receive transducer element Rij, and specifies a receive signal corresponding to a time point after subtraction of the delay amount for fixed receive transducer element Xij from a receive signal sequence corresponding to the fixed receive transducer element Xij.

### <Conclusion>

As described above, the ultrasound diagnostic device pertaining to modification 1 achieves the following effects in addition to the effects described in embodiment 1. In modification 1, in addition to the measurement point signal based on ultrasound reflected from the measurement point Pij received by the receive transducer element Rij, the measurement point signal based on ultrasound reflected from the measurement point Pij received by the fixed receive transducer element Xij is specified, and delay-and-summing of the measurement point signals is performed for multiple transmission events. With respect to the measurement point signal received by the fixed receive transducer element Xij, while a reflected ultrasound receive path from the measurement point Pij to the fixed receive transducer element Xij is fixed, decay of received ultrasound due to the reflected ultrasound receive path is minimum, and thus an S/N ratio is high. Further, the fixed receive transducer element Xij and the receive transducer element Rij that correspond to the measurement point Pij differ from each other for most transmission events. Accordingly, by summing the respective measurement point signal corresponding to the fixed receive transducer element Xij and the receive transducer element Rij for the same transmission event, it is possible to exhibit an effect of an improvement in S/N ratio by delay-and-summing. This further increases the effect of an improvement in S/N ratio.

### Effects of Embodiment 1 and Modification 1

The following describes the effects of embodiment 1 and modification 1 by comparing receive beam forming pertaining to embodiment 1 and modification 1 and two types of receive beam forming that are comparative examples in terms of ultrasound image quality.

### (1) RECEIVE BEAM FORMING IN COMPARATIVE EXAMPLES

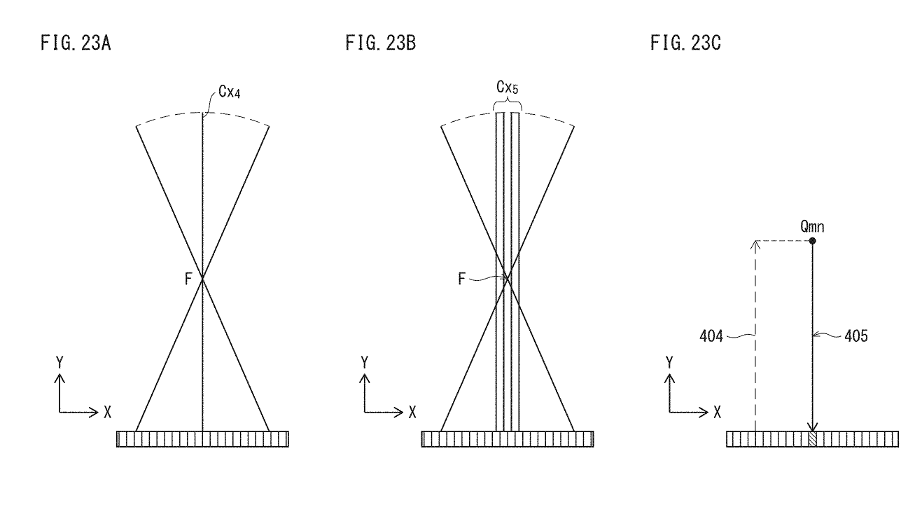

Transmission beam forming of comparative example 1 is the same as those in embodiment 1 and modification 1. Meanwhile, in receive beam forming of comparative example 1, conventional delay-and-summing is performed. Specifically, as illustrated in FIG. 23A, a measurement point is set on a straight line Cx₄ that passes through the transmission focal point F and is perpendicular to the transducer element array direction, and delay-and-summing is performed with respect to ultrasound reflected from the measurement point on the straight line Cx₄, and thereby an acoustic line signal is generated. This means that the receive beam forming of comparative example 1 does not use the synthetic aperture method and performs delay-and-summing for each transmission event. Note that in comparative example 1, the calculation of transmission time may be performed using only measurement point depth. This change in calculation method has no influence on the resulting ultrasound images. This is because, for measurement points on the straight line Cx₄ of comparative example 1, a transmission time calculated according to the present embodiment and a transmission time calculated according to the conventional technique of using only measurement point depth are exactly equal.

Transmission beam forming of comparative example 2 is also the same as those in embodiment 1 and modification 1. Meanwhile, in receive beam forming of comparative example 2, operations similar to those in embodiment 1 and modification 1 are performed. In specific, the fixed receive transducer element Xij is identified by the same method as that in modification 1, and from the receive signal sequence for the fixed receive transducer element Xij, a receive signal corresponding to a time point after subtraction of a delay amount is specified as a measurement point signal based on ultrasound reflected from the measurement point Pij. Further, delay-and-summing of only measurement point signals corresponding to the fixed receive transducer element Xij is performed for transmission events. That is, in comparative example 2, differently from embodiment 1 and modification 1, the receive transducer element Rij is not identified, and a measurement point signal corresponding to the receive transducer element Rij is not specified.

### (2) ULTRASOUND IMAGE QUALITY

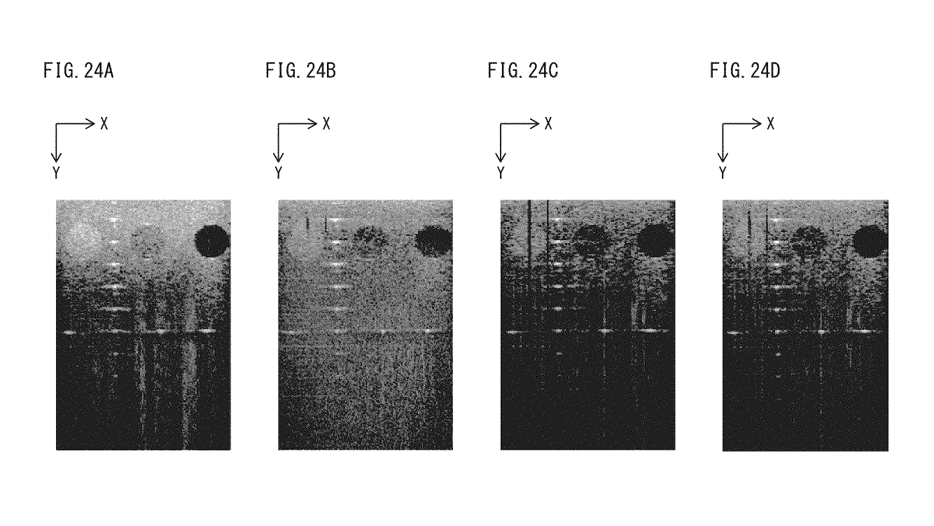

FIGS. 24A through 24D show ultrasound images (B-mode tomographic images) acquired by image-capturing the same imaging phantom by using the receive beam forming methods of embodiment 1 and modification 1 and the receive beam forming methods of comparative examples 1 and 2. Specifically, FIGS. 24A through 24D correspond to comparative example 1, embodiment 1, comparative example 2, and modification 1, respectively. Note that in each of these drawings, the Y direction is the depth direction and the X direction is the transducer element array direction. FIGS. 24A through 24D each illustrate a rectangular region of the same X coordinate range and Y coordinate range that is extracted from the ultrasound image.

As illustrated in FIG. 24A, in comparative example 1, (i) the greater the distance from the transmission focal depth, the greater the bleeding of bright spots, which should have circular shapes, in the X direction, and (ii) the greater the depth, the greater the amount of noise. Especially, the image is unclear in an area that is deeper than a depth at which four bright spots are arrayed in the X direction. These problems are considered to have occurred due to ultrasound beams becoming more out of focus, transmitted ultrasound amplitude decreasing, and phase lag increasing, as distance from the focal point F increases.

Meanwhile, as illustrated in FIG. 24B, in embodiment 1, (i) in an area shallower than the transmission focal depth, bleeding of bright spots in the X direction is greater than in comparative example 1. On the other hand, the amount of noise does not increase even after the depth increases. Especially, the image is still clear even in the area that is deeper than the depth at which four bright spots are arrayed in the X direction. It is considered that this is due to an improvement in spatial resolution by the synthetic aperture method despite a distance from the transmission focal point F, owing to a great diversity of the ultrasound beam transmission path from the transmission aperture Tx to the measurement point Pij and the reflected ultrasound receive path from the measurement point Pij to the receive transducer element Rij. The following two cases can be considered as reasons for improvement in spatial resolution. (i) Since distance resolution and directional resolution vary depending upon ultrasound beam travel direction, spatial resolution and S/N ratio are improved owing to complementation by delay-and-summing of measurement point signals with ultrasound beam transmission path that are greatly different from each other. (ii) Since the ultrasound beam transmission path differs from the reflected ultrasound receive path, difference occurs in terms of pattern of noise influenced by the surrounding of the measurement point, synthesizing cancels out noise, and thus S/N ratio is improved. On the other hand, it is considered that in the area shallower than the transmission focal depth, bleeding of bright spots in the X direction is greater than in comparative example 1, because of an insufficient improvement in S/N ratio due to a small number of measurement point signals to be used for delay-and-summing.

Compared with this, as illustrated in FIG. 24C, in comparative example 2, in the area shallower than the transmission focal depth, the degree of bleeding of bright spots in the X direction is equal to that in embodiment 1, and the image is clearer than in comparative example 1, but is unclearer than in embodiment 1. It is considered that this is because of the following reasons. In specific, the image is clearer than in comparative example 1 because diversity of the ultrasound beam transmission path from the transmission aperture Tx to the measurement point Pij is ensured and spatial resolution is improved by the synthetic aperture method despite a distance from the transmission focal point F. Meanwhile, the image is unclearer than in embodiment 1 because a lower effect of improvement in spatial resolution by the synthetic aperture method is exhibited compared with embodiment 1 due to fixing of the reflected ultrasound receive path from the measurement point Pij to the fixed receive transducer element Xij. Further, in FIG. 24C, several black lines appear on the left side of the bright spots arrayed in the Y direction. This is due to a low receive sensitivity of a specific transducer elements. In comparative example 2, ultrasound reflected from the measurement point Pij is received by only the fixed receive transducer element Xij having the same X coordinates as the measurement point Pij. Thus, there is variation in receive sensitivity of transducer elements. Especially when a specific transducer element has a low receive sensitivity, an area is generated that has a low luminance and is composed of a straight line extending in the Y direction from the transducer element, as illustrated in FIG. 24C.

Meanwhile, as illustrated in FIG. 24D, in modification 1, in the area shallower than the transmission focal depth, the degree of bleeding of the bright spots in the X direction is smaller than in embodiment 1 and comparative example 2. Also, the amount of noise does not increase and the image is still clear even after the depth increases, like in embodiment 1. It is considered that this is because of the following reasons: (i) S/N ratio is increased compared with embodiment 1 and comparative example 2 owing to an increased number of measurement point signals to be used for delay-and summing; and (ii) an effect of an improvement in spatial resolution and S/N ratio by the synthetic aperture method is increased compared with comparative example 2 owing to an increased diversity of the reflected ultrasound receive path from the measurement point Pij to the transducer element. Further, an area with a low luminance appears in the Y direction, but is pale and is not noticeable compared with that in FIG. 24C. This is because even when a specific fixed transducer element Xij has a low receive sensitivity, complementation can be performed with respect to a measurement point Pij having the same X coordinates as the specific fixed transducer element Xij by measurement point signals received by multiple receive transducer elements Rij.

### <Embodiment 2

In the ultrasound diagnostic device 100 pertaining to embodiment 1 and the ultrasound diagnostic device pertaining to modification 1, description is provided of the case where one and two receive transducer elements with respect to each measurement point are identified for each transmission event, respectively, and one and two measurement point signals are specified for the one and two identified receive transducer elements, respectively, and delay-and-summing of the specified measurement point signals is performed for a plurality of transmission events, and thus an acoustic line signal is generated. However, especially in embodiment 1, S/N ratio of acoustic line signals might be insufficiently improved because of a small number of measurement point signals to be used for delay-and-summing (receive signals acquired for a plurality of transmission events on which delay processing has been appropriately performed by the receive beam former using a synthetic aperture method).

An ultrasound diagnostic device pertaining to embodiment 2 generates, in addition to an acoustic line signal pertaining to embodiment 1 or modification 1, a sub-frame acoustic line signal for each transmission event using a conventional synthetic aperture method, and synthesizes the acoustic line signal pertaining to embodiment 1 or modification 1 and the sub-frame acoustic line signals, thereby generating a frame acoustic line signal. This is a difference from embodiment 1.

### <Structure>

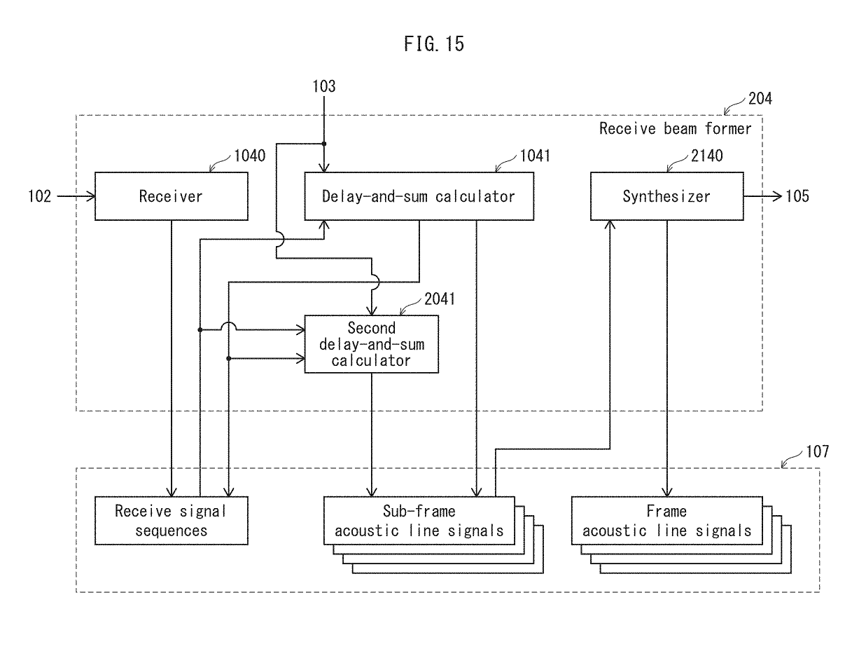

The following describes the ultrasound diagnostic device pertaining to embodiment 2, with reference to the accompanying drawings. FIG. 15 is a functional block diagram illustrating the structure of a receive beam former 204 of the ultrasound diagnostic device pertaining to embodiment 2. As illustrated in FIG. 15, the receive beam former 204 includes, in addition to a receiver 1040, a delay-and-sum calculator 1041, a second delay-and-sum calculator 2041 and a synthesizer 2140.

The following describes the second delay-and-sum calculator 2041 and the synthesizer 2140 among the units constituting the receive beam former 204. Meanwhile, the receiver 1040 and the delay-and-summing calculator 1041 are similar to those in embodiment 1, and thus, description thereof is not provided in the following.

(1) Second Delay-and-Sum Calculator 2041

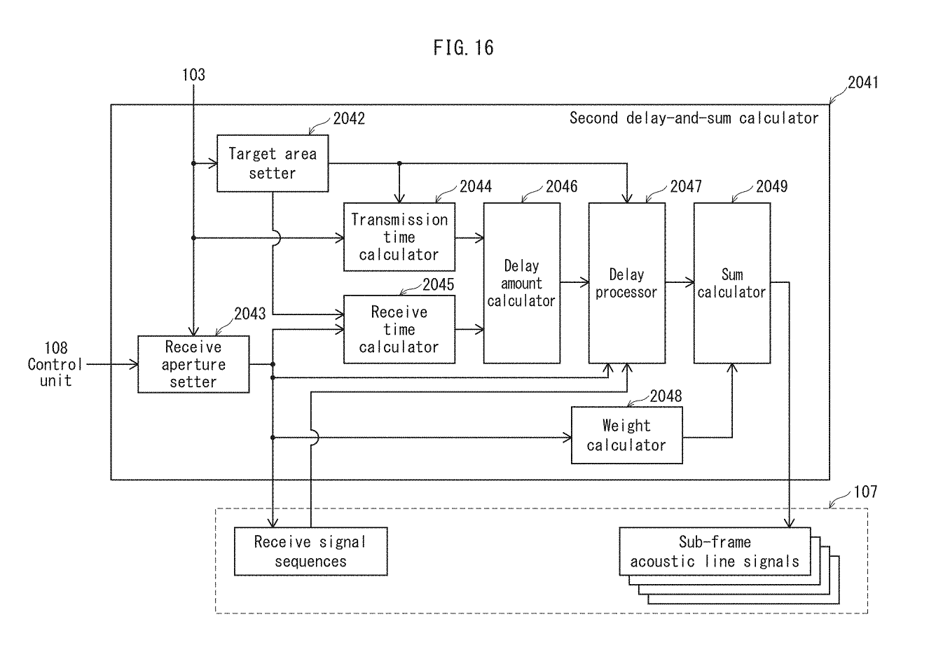

The second delay-and-sum calculator 2041 sets a target area Cx for each transmission event. A target area Cx is an area in the subject from which one sub-frame acoustic line signal is to be generated. Further, the second delay-and-sum calculator 2041 performs, for each measurement point Pij of the target area Cx, delay-and-sum processing with respect to receive signal sequences corresponding to the measurement point Pij, each of which is received by one receive transducer element Rk. The second delay-and-sum calculator 2041 performs this processing for each transmission event having been performed. The second delay-and-sum calculator 2041, for each transmission event, generates a sub-frame acoustic line signal for the transmission event by calculating an acoustic line signal for each measurement point of the target area Cx for the transmission event. FIG. 16 is a functional block diagram illustrating the structure of the second delay-and-sum calculator 2041. As illustrated in FIG. 16, the second delay-and-sum calculator 2041 includes: a target area setter 2042; a receive aperture setter 2043; a transmission time calculator 2044; a receive time calculator 2045; a delay amount calculator 2046; a delay processor 2047; a weight calculator 2048; and a sum calculator 2049.

The following describes the structure of each functional block of the second delay-and-sum calculator 2041.

### i) Target Area Setter 2042

The target area setter 2042 sets the target area Cx, which is an area in the subject from which one sub-frame acoustic line signal is to be generated. More specifically, in the present disclosure, the term “target area” is used to indicate a signal area for generating a sub-frame acoustic line signal for one transmission event. Further, one acoustic line signal is generated for each measurement point Qmn of the target area Cx. In other words, the target area Cx is set for each transmission event in order to specify ones of the measurement points for which acoustic line signals are to be generated for the transmission event.

Further, in the present disclosure, a sub-frame acoustic line signal is a group of acoustic line signals that are generated from one transmission event. As already described above, from one transmission event, a plurality of acoustic line signals are generated, each for a different one of the measurement points Qmn of the target area Cx. Further, a sub-frame is a unit corresponding to a group of signals which are acquired from one transmission event and each of which corresponds to a different one of the measurement points Qmn of the target area Cx for the transmission event. Thus, a synthesizing of multiple sub-frames acquired at different time points equals one frame.

For each transmission event, the target area setter 2042 sets the target area Cx based on the information indicating the position of the transmission aperture Tx for the transmission event, which is acquired from the transmission beam former 103.

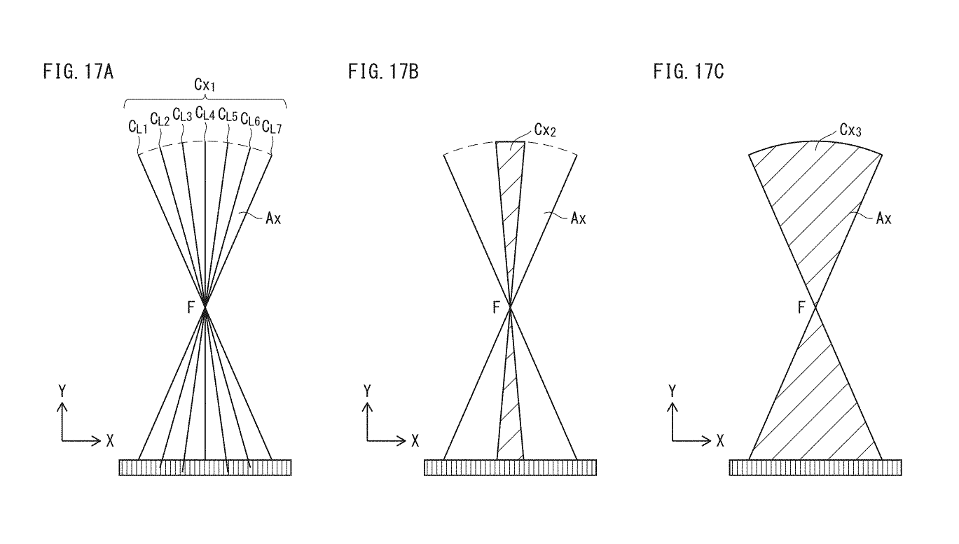

FIG. 17A through FIG. 17C are schematics illustrating the target area Cx. As illustrated in FIG. 17A, a target area Cx₁ is set inside the ultrasound main irradiation area Ax, and is composed of target lines CL₁ through CL₇. Each of the target lines passes through the transmission focal point F or the focal area. Among the target lines CL₁ through CL₇, target lines CL₁ and CL₇ each correspond to an outer boundary of the ultrasound main irradiation area Ax, and target line CL₄ is located on a center axis Txo of the transmission aperture. For the sake of convenience, the following description is provided based on the assumption that the ultrasound main irradiation area Ax has two outer boundaries, one being a straight line passing through the transmission focal point F and one end of the transmission aperture Tx, and the other being a straight line passing through the transmission focal point F and the other end of the transmission aperture Tx. Further, every pair of adjacent ones of the target lines CL₁ through CL₇ form substantially the same angle therebetween. This means that measurement points on an arc centered on the transmission focal point F are located at the same distance from one another. Further, a distance dj between two adjacent measurement points Qmn on the same target line CL₁ is smaller than a distance di between two measurement points Qmn on the adjacent target lines CL₁ and CL₂. Note that distance di is at least twice the distance dj, is preferably at least four times the distance dj, and is more preferably at least eight times the distance dj. This configuration allows arranging measurement points uniformly over substantially the entirety of the ultrasound main irradiation area Ax while making measurement point density in the depth direction high and measurement point density transverse to target lines (substantially similar to the transducer element array direction and a circumferential direction of an arc centered on transmission focal point F) low.

Note that the target area Cx₁ need not have the shape described above. For example, points of the target lines CL₁ through CL₇ coming in contact with the transmission transducer element array may be spaced away at equal distance from one another. Further, while the example of the target area Cx₁ described above is composed of seven target lines, the number of target lines in the target area Cx may be set to any value without being limited to seven.

Further, as other variations of the target area Cx, the target area setter 2042 may set a target area Cx₂ having a narrower width in the X direction than the ultrasound main irradiation area Ax as illustrated in FIG. 17B. Alternatively, the target area setter 2042 may set a target area Cx₃ that equals the entirety of the ultrasound main irradiation area Ax as illustrated in FIG. 17C.

The target area setter 2042 outputs the target area Cx to the transmission time calculator 2044, the receive time calculator 2045, and the delay processor 2047.

### ii) Receive Aperture Setter 2043

The receive aperture setter 2043 is a circuit that sets, for each transmission event, receive apertures Rx based on a control signal from the control unit 108 and information from the transmission beam former 103 indicating a position of a transmission aperture Tx for the transmission event. In specific, the receive aperture setter 2043 selects, for each measurement point Qmn of the target area Cx, some of the transducer elements 101a of the probe 101 as receive transducer elements forming a transducer element array (referred to in the following as a receive transducer element array) whose center position corresponds to a transducer element Xk spatially closest to the measurement point Qmn.

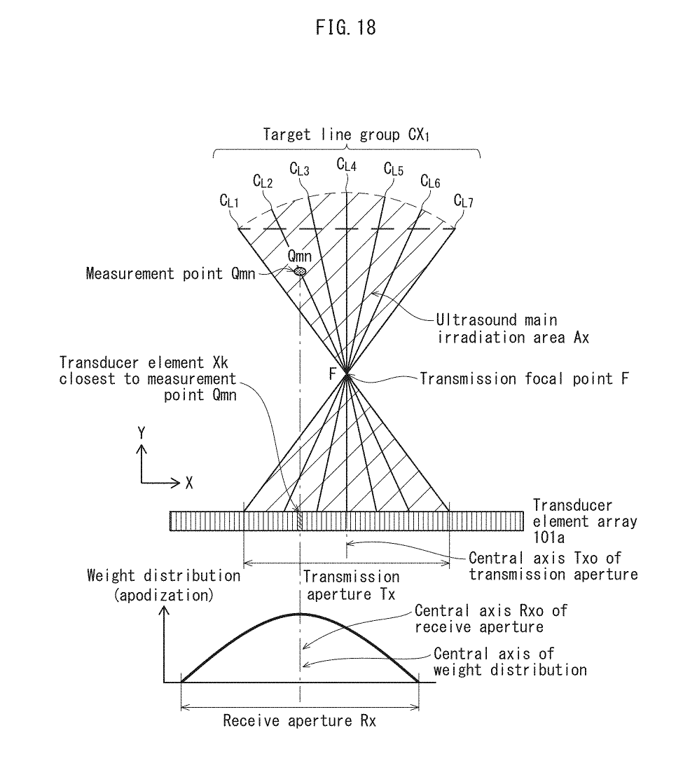

The receive aperture setter 2043 sets, for each measurement point Qmn of the target area Cx for a transmission event, a receive aperture Rx (i.e., the receive transducer element array) so that the center position of the receive aperture Rx in the transducer element array direction corresponds to a transducer element Xk that is spatially closest to the measurement point Qmn. FIG. 18 is a schematic illustrating the relationship between a transmission aperture Tx and a receive aperture Rx that the receive aperture setter 2043 sets. As illustrated in FIG. 18, for a given measurement point Qmn, the receive aperture Rx is set so that the center position of the receive aperture Rx in the transducer element array direction corresponds to a transducer element Xk that is spatially closest to the measurement point Qmn. Due to this, the position of the receive aperture Rx depends upon the position of the measurement point Qmn, and does not change depending upon the position of the transmission aperture Tx, which shifts each time a transmission event is performed. That is, delay-and-sum processing for generating an acoustic line signal for a given measurement point Qmn is always performed based on receive signal sequences acquired by receive transducer elements Rk composing the same receive aperture Rx. This means that with respect to the measurement point Qmn, the same receive aperture Rx is used in delay-and-sum processing irrespective of transmission events.

In order to utilize reflected ultrasound from the entirety of the ultrasound main irradiation area, the number of the receive transducer elements composing each receive aperture Rx is, beneficially, greater than or equal to the number of transmission transducer elements composing each transmission aperture Tx. For example, the number of receive transducer elements may be 32, 64, 96, 128, 192, and so on.

The setting of the receive apertures Rx is performed at least for each transmission event. Due to this, the setting of the receive apertures Rx is repeated at least for the number of times transmission events are performed. Further, the setting of receive apertures Rx may be performed each time a transmission event is performed as described above, or alternatively, receive apertures Rx for multiple transmission events having been performed may be set at once after the completion of the transmission events.

Further, the receive aperture setter 2043 outputs information indicating the positions of the receive transducer elements composing the receive aperture Rx to the data storage 107, via the control unit 108.

The data storage 107 outputs the information indicating the positions of the receive transducer elements composing the receive aperture Rx along with receive signal sequences for the receive transducer elements to each of the transmission time calculator 2044, the receive time calculator 2045, the delay processor 2047, and the weight calculator 2048.

iii) Transmission Time Calculator 2044

The transmission time calculator 2044 is a circuit that, for each transmission event, calculates a transmission time for each measurement point Qmn of the target area Cx for the transmission event. The transmission time for a given measurement point Qmn is the time amount required for transmitted ultrasound to arrive at the measurement point Qmn. The transmission time calculator 2044 acquires information indicating the positions of the transmission transducer elements for a given transmission event from the data storage 107, and information indicating the position of the target area Cx for the transmission event from the target area setter 2042. Based on such information, the transmission time calculator 2044, for each measurement point Qmn located on the target areas composing the target area Cx, calculates the transmission time required for transmitted ultrasound to arrive at the measurement point Qmn. A specific calculation method is the same as that performed by the transmission time calculator 1044 pertaining to embodiment 1, and thus the details thereof are omitted here.

For each transmission event, the transmission time calculator 2044 calculates the transmission time for each measurement point Qmn of the target area Cx for the transmission event. That is, the transmission time calculator 2044 calculates, for each measurement point Qmn, the time amount required for transmitted ultrasound to arrive at the measurement point Qmn. Further, the transmission time calculator 2044 outputs the transmission time so calculated to the delay amount calculator 2046.

### iv) Receive Time Calculator 2045

The receive time calculator 2045 is a circuit that calculates, for each measurement point Qmn, a receive time required for ultrasound reflection from the measurement point Qmn to arrive at each receive transducer element Rk of the receive aperture Rx. For a given transmission event, the receive time calculator 2045 acquires information indicating the positions of the receive transducer elements Rk for the given transmission event from the data storage 107, and acquires the information indicating the position of the target area Cx for the given transmission event from the target area setter 2042. Based on such information, the receive time calculator 2045, for each measurement point Qmn of the target area Cx, calculates the receive time required for transmitted ultrasound to arrive at each receive transducer element Rk after being reflected at the measurement point Qmn. A specific calculation method is the same as that performed by the receive time calculator 1045 pertaining to embodiment 1, and thus the details thereof are omitted here.

For each transmission event, the receive time calculator 2045 calculates the receive time for each measurement point Qmn of the target area Cx for the transmission event. That is, the receive time calculator 2045 calculates, for each measurement point Qmn, the time required for transmitted ultrasound to arrive at each receive transducer element Rk after being reflected at the measurement point Qmn. Further, the receive time calculator 2045 outputs the receive time so calculated to the delay amount calculator 2046.

### v) Delay Amount Calculator 2046

The delay amount calculator 2046 is a circuit that calculates, for each receive transducer element Rk, a total propagation time based on the transmission time and the receive time for the receive transducer element Rk. Further, the delay amount calculator 2046 calculates, for each receive transducer element Rk, a delay amount to be applied to a receive signal sequence for the receive transducer element Rk. In specific, the delay amount calculator 2046 acquires, from the transmission time calculator 2044, the transmission time required for ultrasound waves to arrive at a measurement point Qmn. Further, for each receive transducer element Rk, the delay amount calculator 2046 acquires, from the receive time calculator 2045, the receive time required for ultrasound to be reflected at the measurement point Qmn and arrive at the receive transducer element Rk. Then, the delay amount calculator 2046, for each receive transducer element Rk, calculates a total propagation time required for transmitted ultrasound to arrive at the receive transducer element Rk. Further, based on the difference between total propagation times for the receive transducer elements Rk, the delay amount calculator 2046 calculates a delay amount for each receive transducer element Rk. For each measurement point Qmn of the target area Cx, the delay amount calculator 2046 calculates, for each receive transducer element Rk, the delay amount to be applied to a receive signal sequence for the receive transducer element Rk, and outputs the delay amounts to the delay processor 2047.

### vi) Delay Processor 2047

The delay processor 2047 is a circuit that specifies, for each receive transducer element Rk, a receive signal based on reflected ultrasound from a measurement point Qmn. In specific, for each receive transducer element Rk, the delay processor 2047 specifies a receive signal corresponding to the delay amount for the receive transducer element Rk from the receive signal sequence for the receive transducer element Rk.

More specifically, for each transmission event, the delay processor 2047 acquires, for each receive transducer element Rk, information indicating the position of the receive transducer element Rk from the receive aperture setter 2043, the receive signal sequence for the receive transducer element Rk from the data storage 107, and the delay amount to be applied to the receive signal sequence of the receive transducer element Rk from the delay amount calculator 2046. In addition, for each transmission event, the delay processor 2047 acquires the information indicating the position of the target area Cx from the target area setter 2042. Further, for each receive transducer element Rk, the delay processor 2047 specifies a receive signal based on reflected ultrasound from a measurement point Qmn. In specific, the delay processor 2047 specifies, from the receive signal sequence for the receive transducer element Rk, a receive signal corresponding to a time point after subtraction of the delay amount for the receive transducer element Rk. The delay processor 2047 outputs the receive signal so specified to the sum calculator 2049.

vii) Weight Calculator 2048

The weight calculator 2048 is a circuit that calculates a weight sequence (reception apodization weight) for the receive transducer elements Rk, so that the maximum weight is set with respect to the receive transducer element located at the center of the receive aperture Rx in the transducer element array direction.

As illustrated in FIG. 18, the weight sequence is a numerical sequence of weight coefficients that are to be applied to receive signals for the receive transducer elements composing the receive aperture Rx. The weight sequence indicates weights that are distributed symmetrically with respect to the measurement point Qmn. As the shape of distribution of the weights indicated by the weight sequence, any shape is applicable, including but not limited to a hamming window, a hanning window, and a rectangular window. The weight sequence is set so that the maximum weight is set with respect to the receive transducer element located at the center position of the receive aperture Rx in the transducer element array direction, and the central axis of the weight distribution corresponds to the center axis Rxo of the receive aperture Rx. The weight calculator 2048 uses as input information indicating the positions of the receive transducer elements Rk, which is output from the receive aperture setter 2043, and outputs the weight sequence for the receive transducer elements Rk to the sum calculator 2049.

viii) Sum Calculator 2049

The sum calculator 2049 is a circuit that generates a delayed-and-summed acoustic line signal for each measurement point Qmn, by using as input the specified receive signals for the receive transducer elements Rk, which are output from the delay processor 2047, and summing together the specified receive signals. Alternatively, the sum calculator 2049 may generate an acoustic line signal for each measurement point Qmn by using as input the weight numerical sequence for the receive transducer elements Rk, which is output from the weighting calculator 2048, multiplying the specified receive signal for each receive transducer element Rk with a corresponding weight, and summing the weighted receive signals.

As a result of one transmission event and processing accompanying the transmission event, an acoustic line signal is generated for each measurement point Qmn of the target area Cx for the transmission event. Further, by repetitively performing transmission events while shifting the transmission aperture Tx in the transducer element array direction each time, all of the transducer elements 101a in the probe 101 perform ultrasound transmission. Due to this, a frame acoustic line signal, which is a synthesizing of acoustic line signals corresponding to one frame, is generated.

In the present embodiment, acoustic line signals for respective measurement points, which compose the frame acoustic line signal and each of which is generated by synthesizing a plurality of acoustic line signals corresponding to the measurement point that are included in different sub-frame acoustic line signals, are each referred to as a synthesized acoustic line signal for the measurement point.

The sum calculator 2049, for each transmission event, generates a sub-frame acoustic line signal being a synthesizing of acoustic line signals for every measurement point Qmn of the target area Cx for the transmission event. Further, the sum calculator 2049 outputs the sub-frame acoustic line signals so generated to be stored in the data storage 107.

### (2) Synthesizer 2140

The synthesizer 2140 is a circuit that generates a frame acoustic line signal by synthesizing an acoustic line signal generated by the delay-and-sum calculator 1041 and sub-frame acoustic line signals each generated for one transmission event by the second delay-and-sum calculator 2041.

The synthesizer 2140, after the generation of a series of sub-frame acoustic line signals necessary for generating one frame acoustic line signal is completed, reads out the acoustic line signal generated by the delay-and-sum calculator 1041 and the sub-frame acoustic line signals from the data storage 107. Further, the synthesizer 2140 generates a frame acoustic line signal by synthesizing the acoustic line signal and the sub-frame acoustic line signals. The synthesizing of the acoustic line signal and the sub-frame acoustic line signals is performed according to the positions of the measurement points, such that in the process, a synthesized acoustic line signal is generated for each measurement point. In specific, the synthesizer 2140 generates a synthesized acoustic line signal for a given measurement point by synthesizing a plurality of acoustic line signals corresponding to the measurement point. Due to this, acoustic line signals for the same measurement point that are included in different sub-frame acoustic line signals are synthesized, to generate a synthesized acoustic line signal for the measurement point.

Further, for a measurement point included in multiple target areas Bx and Cx, values of a plurality of acoustic line signals included in different sub-frame acoustic line signals are summed. Thus, the synthesized acoustic line signal for such a measurement point may indicate a great value, depending upon the number of target areas Bx and Cx in which the measurement point is included. In order to moderate this, the synthesizer 2140, in synthesizing the acoustic line signals to generate the frame acoustic line signal, performs amplification of multiplying each of the synthesized acoustic line signals by an amplification factor. The amplification factor is determined according to the number of acoustic line signals synthesized to yield to the synthesized acoustic line signal. Specific processing is the same as that performed by the amplifier 1049 pertaining to embodiment 1, and thus the details thereof are omitted.

Here, note that the synthesizer 2140 may generate the frame acoustic line signal by synthesizing amplified synthesized acoustic line signals for respective measurement points.

### <Operations>

The following describes the operations of the ultrasound diagnostic device pertaining to embodiment 2 having the structure described up to this point.

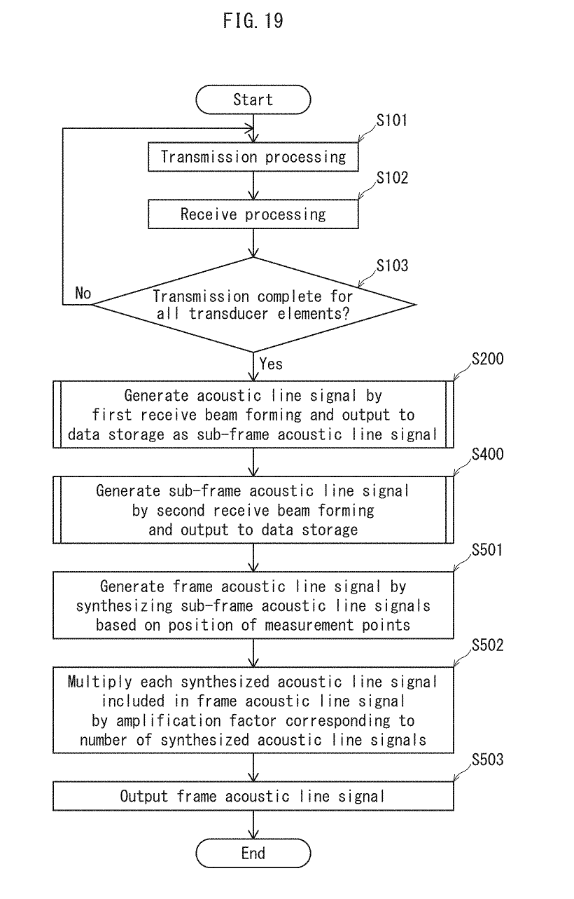

FIG. 19 is a flowchart illustrating beam forming by the receive beam former 204. Note that the same operations as those in the steps of the flowcharts in FIG. 8 and FIG. 9 have the same step numbers as those in FIG. 8 and FIG. 9, and the details thereof are omitted.

First, in Step S101, the receive beam former 204 performs transmission events. In Step S102, the receive beam former 204 performs receive processing for each transmission event. The processing in Steps S101 and S102 is repeatedly performed by shifting the transmission aperture Tx in the transducer element array direction by the width of a single transducer element until completion of ultrasound transmission from all the transducer elements 101a of the probe 101 (Step S103).

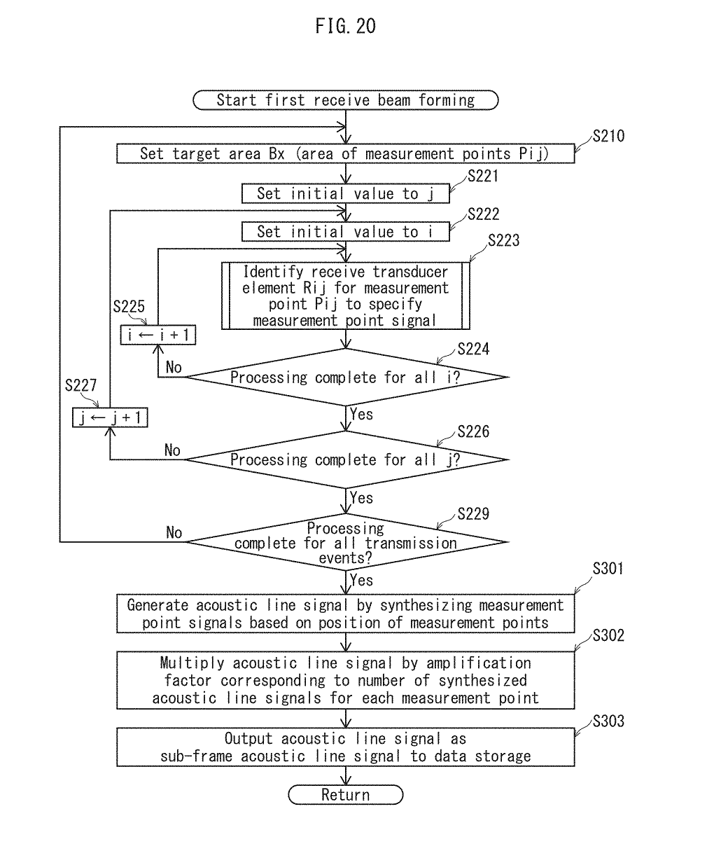

Subsequently, in Step S200, the receive beam former 204 performs first beam forming to generate an acoustic line signal, and outputs the acoustic line signal as a sub-frame acoustic line signal to the data storage 107. FIG. 20 is a flowchart illustrating first beam forming by the delay-and-sum calculator 104.

First, in Step S210, the target area setter 1042 sets a target area Bx for a processing-target transmission event based on information indicating the position of the transmission aperture Tx for the processing-target transmission event.

Subsequently, coordinate values i and j indicating a position of a measurement point Pij are initialized to the respective minimum possible values in the target area Bx (Steps 5221 and S222).

Subsequently, a measurement point signal is specified for the measurement point Pij (Step S223).

Subsequently, a measurement point signal is specified for each measurement point Pij on the coordinate values i and j of the target area Bx by repeating Step S223 while incrementing the coordinate values i and j.

Subsequently, a determination is performed of whether or not a measurement point signal has been specified for every transmission event (Step S229). When the measurement point signal has not yet been specified for every transmission event, processing proceeds to Step S210, where the coordinate values i and j indicating the position of the measurement point Pij are initialized to the respective minimum possible values in the target area Bx for the subsequent transmission event, which can be calculated from the transmission aperture Tx for the subsequent transmission event (Steps S221 and S222), and then specification of a measurement point signal is performed (Step S223). Meanwhile, when the measurement point signal has been specified for every transmission event having been performed, processing proceeds to Step S301.

Subsequently, in Step S301, the adder 1048 synthesizes the measurement point signals based on the positions of the measurement points Pij to generate an acoustic line signal for each measurement point Pij. Subsequently, the amplifier 1049 multiplies each acoustic line signal by a corresponding amplification factor that is determined based on the number of measurement point signals synthesized to yield the acoustic line signal (Step S302). Further, the amplifier 1049 outputs the amplified acoustic line signals as sub-frame acoustic line signals to the data storage 107 (Step S303), and processing is terminated.

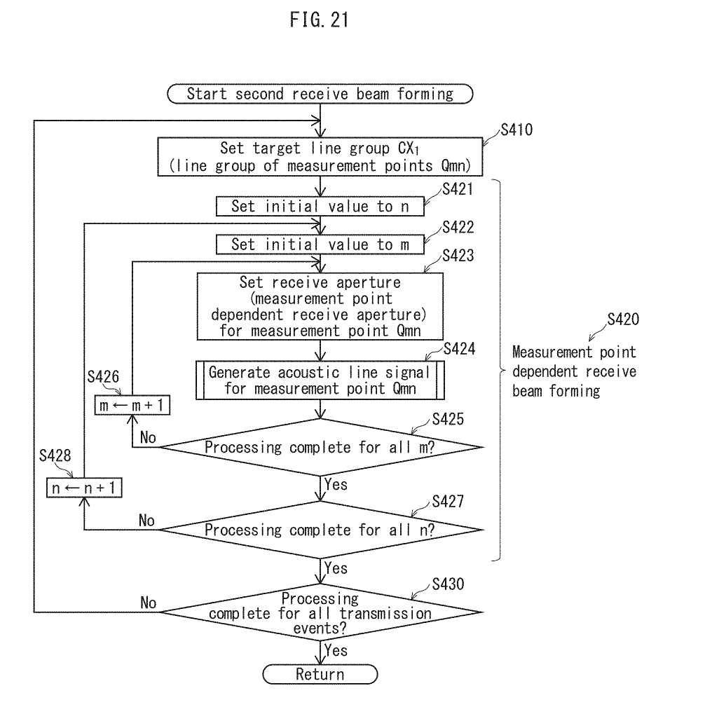

Returning to FIG. 19 once again, subsequently in Step S400, the receive beam former 204 performs second beam forming to generate a sub-frame acoustic line signal, and outputs the sub-frame acoustic line signal to the data storage 107. FIG. 21 is a flowchart illustrating second beam forming by the second delay-and-sum calculator 2041.

First, in Step S410, the target area setter 2042 sets a target area Cx based on information indicating a position of a transmission aperture Tx for each transmission event. In the initial loop of processing, the target area setter 2042 sets a target area Cx for the initial transmission event, which can be calculated from the transmission aperture Tx for the initial transmission event.

Subsequently, processing proceeds to measurement point dependent receive beam forming (Step S420). In Step S420, first, coordinate values m and n indicating a position of a measurement point Qmn of the target area Cx for the processing-target transmission event are initialized (set to the respective minimum possible values in the target area Cx) (Steps S421 and S422). Then, the receive aperture setter 2043 sets a receive aperture Rx for the current measurement point so that the center of the receive aperture Rx corresponds to a transducer element Xk that is spatially closest to the current measurement point Qmn (Step S423).

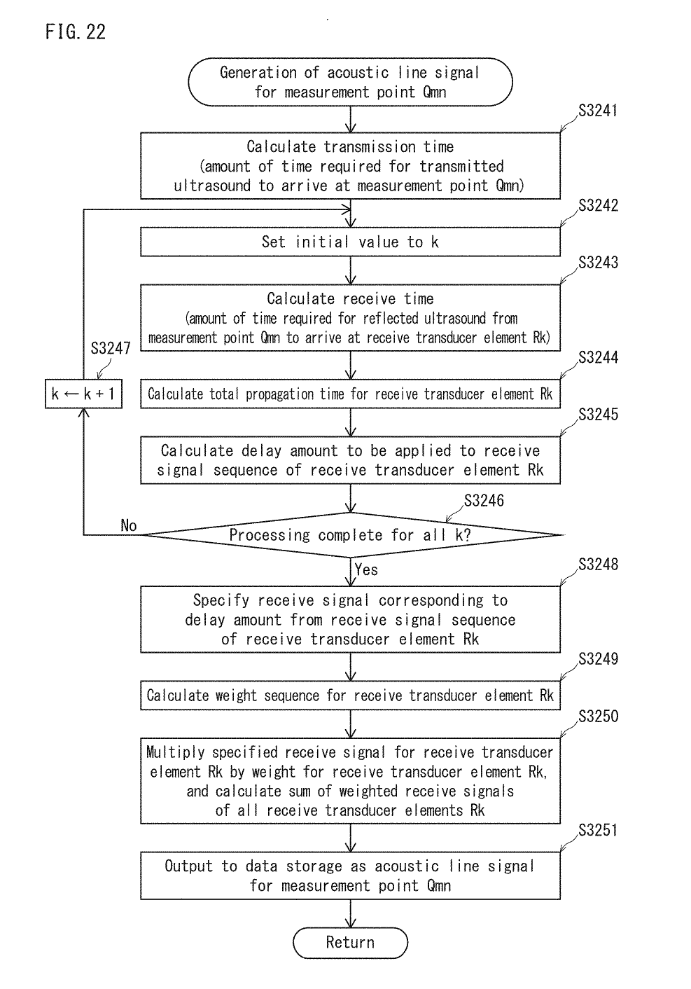

The following describes the operations in Step S423 for generating an acoustic line signal for the current measurement point Qmn. FIG. 22 is a flowchart illustrating the operations of the second delay-and-sum calculator 2041 for generating the acoustic line signal for the current measurement point Qmn.

First, in Step S3241, the transmission time calculator 2044 calculates, for the current measurement point Qmn, a transmission time required for transmitted ultrasound to arrive at the current measurement point Qmn. As already described above, the current measurement point Qmn is a measurement point of the target area Cx for the processing-target transmission event.

Subsequently, in Step S3242, value k, which indicates the position of a target receive transducer element Rk of the receive aperture Rx, is initialized (set to the minimum possible value in the receive aperture Rx) (Step S3242). Then, the receive time for the target receive transducer element Rk is calculated (Step S3243). The receive time is the time required for transmitted ultrasound to arrive at the target receive transducer element Rk after being reflected at the current measurement point Qmn. Further, from a sum of the transmission time and the receive time for the target receive transducer element Rk, the total propagation time required for ultrasound transmitted from the transmission aperture Tx to arrive at the target receive transducer element Rk after being reflected at the current measurement point Qmn is calculated (Step S3244). Further, based on the difference in total propagation time between different receive transducer elements Rk composing the receive aperture Rx, the delay amount for the target receive transducer element Rk is calculated (Step S3245).

Subsequently, a determination is performed of whether or not a delay amount has been calculated for every receive transducer element Rk composing the receive aperture Rx (Step S3246). When a delay amount has not yet been calculated for one or more of the receive transducer elements Rk, the value k is incremented (Step S3247), and a delay amount for another receive transducer element Rk is calculated (Step S3243). Meanwhile, when a delay amount has been calculated for every receive transducer element Rk composing the receive aperture Rx, processing proceeds to Step S3248. Note that at this point, a delay amount for the current measurement point Qmn has already been calculated for each receive transducer element Rk of the receive aperture Rx. The delay amount for a given receive transducer element Rk indicates delay with which reflected ultrasound from the current measurement point Qmn arrives at the receive transducer element Rk.

In Step S3248, the delay processor 2047, for each receive transducer element Rk, specifies a receive signal based on reflected ultrasound from the current measurement point Qmn. Here, the delay processor 2047 specifies, from a receive signal sequence corresponding to each receive transducer element Rk, a receive signal corresponding to a time point after subtraction of the delay amount for the receive transducer element Rk.

Subsequently, the weight calculator 2048 calculates a weight sequence for the receive transducer elements Rk of the current receive aperture Rx, so that the maximum weight is set with respect to the receive transducer element located at the center position of the receive aperture Rx in the transducer element array direction (S3249). Then, the sum calculator 2049 generates an acoustic line signal for the current measurement point Qmn by multiplying the specified receive signal for each receive transducer element Rk by a weight corresponding to the receive transducer element Rk, and summing the weighted receive signals for the different receive transducer elements Rk (Step S3250). Following this, the sum calculator 2049 outputs the acoustic line signal for the current measurement point Qmn to the data storage 107 to be stored in the data storage 107 (Step S3251).

Referring to FIG. 21 once again, subsequently, an acoustic line signal is generated for each measurement point Qmn of the target area Cx for the processing-target transmission event, by repeating Step 424 while incrementing the coordinate values m and n. Subsequently, a determination is performed of whether or not an acoustic line signal has been generated for every measurement point Qmn of the target area Cx (Steps S425 and S427). When an acoustic line signal has not yet been generated for every measurement point Qmn of the target area Cx, the coordinate values m and n are incremented (Steps S426 and S428), yielding an acoustic line signal for another measurement point Qmn (Step S424). Meanwhile, when an acoustic line signal has already been generated for every measurement point Qmn of the target area Cx, processing proceeds to Step S430. At this point, an acoustic line signal has already been generated for each measurement point Qmn of the target area Cx corresponding to the processing-target transmission event, and the acoustic line signals have been output to and stored to the data storage 107. In other words, a sub-frame acoustic line signal for the processing-target transmission event has been generated, and output to and stored to the data storage 107.

Subsequently, a determination is performed of whether or not a sub-frame acoustic line signal has been generated for each transmission event having been performed (Step S430). When sub-frame acoustic line signals have not yet been generated for one or more transmission events, processing proceeds to Step S410, where the coordinate values m and n are initialized (set to the respective minimum possible values in the target area Cx for the subsequent transmission event, which can be calculated from the transmission aperture Tx for the subsequent transmission event) (Steps S421 and S422), and then setting of a receive aperture Rx is performed (Step S423). Meanwhile, when sub-frame acoustic line signals have been generated for every transmission event having been performed, processing proceeds to Step S301.

Subsequently, returning to FIG. 20 once again, in Step S301, the synthesizer 2140 reads out the sub-frame acoustic line signals stored in the data storage 107, and synthesizes the sub-frame acoustic line signals based on positions of the measurement points Pij and Qmn. Thus, a synthesized acoustic line signal is generated for each measurement point Pij and Qmn, and accordingly, a frame acoustic line signal is generated. Subsequently, the synthesizer 2140 multiplies each synthesized acoustic line signal by a corresponding amplification factor that is determined based on the number of acoustic line signals, included in the sub-frame acoustic line signals, that have been synthesized to yield the synthesized acoustic line signal (Step S302). Further, the synthesizer 2140 outputs the amplified frame acoustic line signal to the ultrasound image generator 105 and the data storage 107 (Step S303), and processing is terminated.

### <Conclusion>

As described above, the ultrasound diagnostic device pertaining to embodiment 2 synthesizes an acoustic line signal generated by the delay-and-sum calculator and sub-frame acoustic line signals generated by the second delay-and-sum calculator using a conventional synthesized aperture method. Due to this, it is possible to improve spatial resolution and S/N ratio of frame acoustic line signals owing to the sub-frame acoustic line signals generated by the second delay-and-sum calculator.

Further, computation amount by the delay-and-sum calculator is smaller than that by the second delay-and-sum calculator. Accordingly, the number of measurement points Qmn of the target area Cx exercises a great influence on the computation amount with respect to sub-frame acoustic line signals generated by the second delay-and-sum calculator. Meanwhile, the number of measurement points Pij of the target area Bx exercises little influence on the computation amount with respect to acoustic line signals generated by the delay-and-sum calculator. Due to this, it is possible to greatly reduce the computation amount by setting the target area Bx to the entirety of the ultrasound main irradiation area Ax and setting the target area Cx to part of the target area Bx. On the other hand, it is possible to complement, with the acoustic line signals generated by the delay-and-sum calculator, a decrease in spatial resolution and S/N ratio due to reduction in size or density of the target area Cx. This realizes both improvement in spatial resolution and S/N ratio of frame acoustic line signals and reduction in computation amount.

Accordingly, the ultrasound diagnostic device pertaining to embodiment 2 considerably reduces computation amount compared to ultrasound diagnostic devices using conventional synthetic aperture methods while suppressing degradation of acoustic line signal quality, and contributes to a reduction of processor cost.

### <<Modification 2>>

In the ultrasound diagnostic device pertaining to embodiment 2, the second delay-and-sum calculator generates sub-frame acoustic line signals using a conventional synthetic aperture method. However, the second delay-and-sum calculator may have any structure as long as delay-and-summing can be performed for each transmission event, and is not limited to use the synthetic aperture method.

In modification 2, description is provided of a case where the second delay-and-sum calculator performs receive beam forming by a conventional delay-and-summing that is different from the synthetic aperture method.

FIG. 23A through FIG. 23C are schematics illustrating a setting method of a target area Cx and a calculation method of a transmission time pertaining to modification 2. In modification 2, the target area Cx is composed of one to several straight lines that pass through a transmission focal point F or the vicinity thereof and are perpendicular to the transducer element array direction. For example, in FIG. 23A, a target area Cx₄ is composed of one straight line that passes through the transmission focal point F and is perpendicular to the transducer element array direction. Further, in FIG. 23B, a target area Cx₅ is composed of four straight lines that pass through the vicinity of the transmission focal point F and are perpendicular to the transducer element array direction. Note that the target area Cx₅ is symmetric with respect to one straight line that passes through the transmission focal point F and is perpendicular to the transducer element array direction. Further, in two consecutive transmission events, the transmission focal point F is shifted in the X direction such that eight straight lines constituting two target areas Cx₅ are arrayed at equal intervals in the X direction. In each of these cases, no measurement point Qmn is included in the target area Cx for one frame relating to two different transmission events. Thus, in order to generate an acoustic line signal by synthesizing sub-frame acoustic line signals, summing of sub-frame acoustic line signals based on the positions of the measurement points is performed using only the sub-frame acoustic line signals generated by the second delay-and-sum calculator and the acoustic line signal generated by the delay-and-sum calculator.

Note that a transmission time may be calculated by a conventional method such as illustrated in FIG. 23C. In specific, a depth at a position of a transducer element for a measurement point Qmn is set to zero, a depth y of the measurement point Qmn is calculated, and the depth y is divided by ultrasound velocity cs. Thus, the transmission time can be calculated.

In modification 2, synthesizing is performed with such a method, using acoustic line signals generated by the second delay-and-sum calculator using performing a conventional delay-and-summing and an acoustic line signal generated by the delay-and-sum calculator. Accordingly, it is possible to improve S/N ratio of synthesized acoustic line signals by the acoustic line signals generated by the second delay-and-sum calculator.

Further, the acoustic line signals generated by the second delay-and-sum calculator have different characteristics from the acoustic line signal generated by the delay-and-sum calculator. In specific, with respect to the acoustic line signal generated by the second delay-and-sum calculator, while S/N ratio is high around the transmission focal point F, spatial resolution decreases as a distance of the measurement point Qmn from the transmission focal point F increases. On the other hand, with respect to the acoustic line signal generated by the delay-and-sum calculator, spatial resolution is high irrespective a distance between the measurement point Pij and the transmission focal point F. Thus, synthesizing of these acoustic line signals improves the S/N ratio around the transmission focal point F and the overall spatial resolution.

Further, the computation amount by the second delay-and-sum calculator pertaining to modification 2 is considerably smaller than that in embodiment 2. Thus, it is possible to greatly reduce the computation amount.

### <<Other Modifications>>

(1) In embodiment 1, description is provided of the case where delay-and-summing of measurement point signals is performed without weighting. Alternatively, weighting of measurement point signals may be performed. As weight coefficients for weighting, a numerical sequence of weight coefficients is used so that the maximum weight is set with respect to the transducer element located at the center of the receive aperture, namely, the fixed received transducer element Xij described in modification 1. The weight sequence indicates weights that are distributed symmetrically with respect to the transmission focal point F. As the shape of distribution of the weights indicated by the weight sequence, any shape is applicable, including but not limited to a hamming window, a hanning window, and a rectangular window.

Further, in modification 1, weighting may be performed on the measurement point signal for each receive transducer element Rij and the measurement point signal for the fixed receive transducer element Xij.

(2) In embodiment 2 and modification 2, the delay-and-sum calculator performs delay-and-summing using an acoustic line signal for each receive transducer element Rij. Alternatively, the delay-and-sum calculator may perform delay-and-summing further using a measurement point signal for the fixed receive transducer element Xij, like in modification 1.

(3) In embodiment 2 and modification 2, description is provided of the case where synthesizing of sub-frame acoustic line signals is performed without weighting acoustic line signals generated by the second delay-and-sum calculator and an acoustic line signal generated by the delay-and-sum calculator. Alternatively, the synthesizer may perform weighting. For example, by synthesizing only acoustic line signals generated by the second delay-and-sum calculator and amplifying the weighted acoustic line signals, and then further synthesizing an acoustic line signal generated by the delay-and-sum calculator, it is possible to weight the acoustic line signals generated by the second delay-and-sum calculator and the acoustic line signal generated by the delay-and-sum calculator.

Further, weight coefficients may be varied based on the overlap count or the depth of the measurement point. For example, in embodiment 2, with respect to a measurement point whose overlap count is equal to or higher than a threshold value, it is possible to increase weights for sub-frame acoustic line signals acquired with a high quality by setting a weight coefficient for an acoustic line signal generated by the delay-and-sum calculator to a small value or zero. With respect to a measurement point whose overlap count is less than the threshold value, it is possible to improve quality of frame acoustic line signals by an acoustic line signal generated by the delay-and-sum calculator by increasing a weight coefficient for the acoustic line signal generated by the delay-and-sum calculator. Alternatively, for example, in modification 2, a weight coefficient for an acoustic line signal generated by the delay-and-sum calculator may be set to a small value or zero with respect to an area around the transmission focal depth and may be set to a large value with respect to other areas. With this structure, it is possible to generate frame acoustic line signals, in the area around the transmission focal depth based on an acoustic line signal generated by performing conventional delay-and-summing with a high quality, and in the other areas based on an acoustic line signal generated by the delay-and-sum calculator according to which no decrease in spatial resolution occurs despite a distance from the transmission focal point.

(4) In embodiment 2, the delay-and-sum calculator and the second delay-and-sum calculator are separately configured. Alternatively, the delay-and-sum calculator and the second delay-and-sum calculator may have mostly common structures. For example, the delay-and-sum calculator and the second delay-and-sum calculator differ from each other only in terms of set ranges of measurement points Pij and Qmn, and perform common processing by the transmission time calculator, the receive time calculator, and the delay amount calculator. Accordingly, these calculation units can be shared between the delay-and-sum calculator and the second delay-and-sum calculator. As described above, the entirety of the ultrasound main irradiation area Ax may be set to the target area Bx, and further, all or part of the target area Bx may be set to the target area Cx. Thus, in such a case, the measurement point Qmn may be part of a group of measurement points Pij. Due to this, in the delay-and-sum calculator and the second delay-and-sum calculator, it is possible to perform common computation by the transmission time calculator, the receive time calculator, and the delay amount calculator, thereby further contributing to reduction in computation amount. In addition, with respect to a measurement point for which a sub-frame acoustic line signal is generated, an acoustic line signal is also to be generated inevitably, and this contributes to improvement in spatial resolution and S/N ratio of frame acoustic line signals by synthesizing.

(5) The receive aperture setter 2043 in embodiment 2 selects, for each measurement point Qmn, the receive aperture Rx so that the center position of the receive aperture in the transducer element array direction corresponds to the transducer element Xk that is spatially closest to the measurement point Qmn. However, the structure of the receive aperture Rx may be appropriately modified.

For example, a receive aperture Rx may be set for each transmission event so that the center position of the receive aperture Rx in the transmission element array direction corresponds to the center position of the transmission aperture Tx for the transmission event. In this case, the position of an axis Rxo passing through the center position of the receive aperture Rx corresponds to the position of an axis Txo passing through the center position of the transmission aperture Tx. Further, the receive aperture Rx is symmetric about the transmission focal point F (i.e., has the same number of apertures at both sides of the center position thereof in the transmission element array direction). As such, as the transmission aperture Tx shifts in the transducer element array direction from one transmission event to another, the receive aperture Rx also shifts in the transducer element array direction, following the transmission aperture Tx. In addition, a weight sequence (so-called reception apodization weight) for the receive transducer elements Rk is calculated, so that the maximum weight is set with respect to the receive transducer element Rk located along the center axis Rxo of the receive aperture Rx and the center axis Txo of the transmission aperture Tx. The weight sequence indicates weights distributed symmetrically with respect to the transducer element Xk. As the shape of distribution of the weights indicated by the weight sequence, any shape is applicable, including but not limited to a hamming window, a hanning window, and a rectangular window. Due to this structure, the position of the central axis Rxo of the receive aperture Rx for a given transmission event corresponds to the position of the central axis Txo of the transmission aperture Tx for the same transmission event. Further, when transmission events are repetitively performed, the transmission aperture Tx shifts in the transducer element array direction each time, and the receive aperture Rx also shifts in the transducer element array direction in synchronization with the transmission aperture Tx. Thus, a different receive aperture is used to perform delay-and-sum for each transmission event. Accordingly, receive processing with respect to multiple transmission events can be performed by using a group of receive apertures covering a vast measurement area and each differing in terms of time. Thus, uniform spatial resolution is achieved over a vast measurement area.

(6) Up to this point, the present invention has been described based on specific embodiments and modifications thereof. However, the embodiments and modifications described above are non-limiting examples of application of the present invention, and thus, the present invention shall be construed to encompass the following exemplar modifications.

For example, the present invention may be implemented by using a computer system including a memory storing a computer program and a microprocessor operating based on the computer program. For example, the computer system may store a computer program of the ultrasound signal processing method pertaining to the present invention, and the computer system may operate in accordance with the computer program or may provide instructions in accordance with the computer program to various components connected thereto.

Further, the present invention may be implemented by implementing a part of or the entirety of the ultrasound signal processing device described above, or a part of or an entirety of a beam former described above by using a computer system including a microprocessor, a recording medium such as a ROM or a RAM, and a hard disk unit. In this implementation, a computer program achieving the same operations as a device described above is stored to the RAM or the hard disk unit. Further, in this implementation, various devices achieve their functions by the microprocessor operating in accordance with the computer program.

Further, the present invention may be implemented by implementing some or all components included in a device described above by using one system LSI (large scale integration). A system LSI is an ultra-multifunctional LSI manufactured by integrating multiple components onto one chip. Specifically, a system LSI is a computer system including a microprocessor, a ROM, a RAM, and the like. Further, each component may be separately implemented by using one chip, or some or all components may be implemented by using one chip. Note that LSIs are referred to by using different names, depending upon the level of integration achieved thereby. Such names include IC, system LSI, super LSI, and ultra LSI. In this implementation, a computer program achieving the same operations as any device described above is stored to the RAM. Further, in this implementation, the system LSI achieves its functions by the microprocessor operating in accordance with the computer program. For example, the present invention encompasses a form of implementation where an LSI stores a beam forming method pertaining to the present invention as a program, the LSI is inserted into a computer, and the computer executes the program (i.e., the beam forming method pertaining to the present invention).

Note that integration of circuits may be achieved by a dedicated circuit or a general purpose processor, in addition to being achievable by using an LSI as discussed above. Further, a Field Programmable Gate Array (FPGA), which is programmable after manufacturing, or a reconfigurable processor, which allows reconfiguration of the connection and setting of circuit cells inside the LSI, may be used.

Furthermore, if technology for circuit integration that replaces LSIs emerges, owing to advances in semiconductor technology or to another derivative technology, the integration of functional blocks may naturally be accomplished using such technology.

Further, some or all functions of an ultrasound diagnostic device discussed in the embodiments may be implemented by a processor such as a CPU executing a program. Further, the present invention may be implemented by using a non-transitory computer-readable recording medium having recorded thereon a program causing execution of a diagnostic method and a beam forming method of an ultrasound diagnostic device. Further, execution of the program by another independent computer system may be achieved by transferring the program by recording the program or a signal onto a recording medium. Naturally, the program may be distributed via means of transmission media such as the internet.

The ultrasound diagnostic devices pertaining to the embodiments include the data storage, which is a recording device. However, the recording device need not be included in the ultrasound diagnostic device, and may be implemented by using a semiconductor memory, a hard disk drive, an optical disk drive, a magnetic storage device, or the like connected to the ultrasound diagnostic device from the outside.

Further, the functional blocks illustrated in the block diagrams are mere examples of possible functional blocks. That is, a plurality of functional blocks illustrated in the block diagrams may be combined to form one functional block, a given functional block illustrated in the block diagrams may be divided into a plurality of functional blocks, and a function of a given functional block illustrated in the block diagrams may be transferred to another functional block. Further, with regards to multiple functional blocks having similar functions, such functional blocks may be implemented by one piece of hardware or software executing such functions in parallel or by applying time division.

Further, the above-described order in which steps of processing are executed is a non-limiting example among multiple possible orders that is used for the sole sake of providing specific description of the present invention. Further, some of the steps of processing described above may be executed simultaneously (in parallel).

Further, in the embodiments, description is provided that the ultrasound diagnostic device may have a probe and a display attached thereto. However, the ultrasound diagnostic device may include a probe and a display therein.

Further, in the embodiments, the probe includes a plurality of piezoelectric transducer elements forming a line in one direction. However, the probe may have a different structure. For example, the probe may include a plurality of piezoelectric transducer elements disposed two-dimensionally. Alternatively, the probe may be a swingable probe including a plurality of swingable transducer elements (i.e., transducer elements that can be caused to swing by mechanical means) forming a line in one direction, which enables acquisition of three-dimensional tomographic images. Further, probes of different types may be selected and used depending upon the examination to be performed. For example, when using a probe including piezoelectric transducer elements disposed two-dimensionally, supplying different piezoelectric transducer elements with voltages at different timings or with voltages with different values achieves controlling the position, the direction, etc., of the ultrasound beam to be transmitted.

Further, the probe may be provided with some of the functions of the transmission beam former/receive beam former. For example, the probe may be capable of generating a transmission electric signal based on a control signal that the transmission beam former/receive beam former outputs to cause generation of a transmission electric signal, and of converting the transmission electronic signal into ultrasound. In addition, the probe may be capable of converting reflected ultrasound into a receive electric signal, and of generating a receive signal based on the receive electric signal.

Further, at least some of the functions of the ultrasound diagnostic devices pertaining to the embodiments and the modifications may be combined with functions of other ones of the ultrasound diagnostic devices pertaining to the embodiments and the modifications. Further, the values used above are non-limiting examples used for the sole sake of providing specific description of the present invention, and may be replaced with other values.

Further, the present invention should be construed as encompassing various modifications that a skilled artisan would arrive at based on the embodiments describe above.

### <<Summary>>

(1) One aspect of the present invention is an ultrasound signal processing device that performs multiple transmission events of transmitting converging ultrasound beams to a subject by using an ultrasound probe having multiple transducer elements, and that receives ultrasound reflection from the subject for each of the transmission events to generate sequences of receive signals, and synthesizes sequences of receive signals generated for the respective transmission events to generate an acoustic line signal, the ultrasound signal processing device comprising ultrasound signal processing circuitry configured to operate as: a transmitter that varies a focal point defining a position where ultrasound beams converge between a plurality of transmission events and performs each of the transmission events by causing the ultrasound probe to transmit ultrasound beams directed to an inside of the subject; a receiver that generates, for each of the transmission events, sequences of receive signals for the respective transducer elements of the ultrasound probe based on ultrasound reflection that the ultrasound probe receives from a target area of the subject; and a delay-and-sum calculator that generates an acoustic line signal with respect to each of measurement points of the target area, by performing processing for specifying one measurement point signal for each of the transmission events and performing weighted delay-and-summing of measurement point signals specified for the respective transmission events, the processing including identification of one of the transducer elements that is located on a straight line passing through the measurement point and the focal point and specification of, as the measurement point signal, a receive signal corresponding to the identified transducer element from among the sequence of receive signals for the identified transducer element.

Another aspect of the present invention is an ultrasound signal processing method in which multiple transmission events of transmitting converging ultrasound beams to a subject are performed by using an ultrasound probe having multiple transducer elements, and in which ultrasound reflection is received from the subject for each of the transmission events to generate sequences of receive signals, and sequences of receive signals generated for the respective transmission events are synthesized to generate an acoustic line signal, the ultrasound signal processing method comprising: varying a focal point defining a position where ultrasound beams converge between a plurality of transmission events and performing each of the transmission events by causing the ultrasound probe to transmit ultrasound beams directed to an inside of the subject; generating, for each of the transmission events, sequences of receive signals for the respective transducer elements of the ultrasound probe based on ultrasound reflection that the ultrasound probe receives from a target area of the subject; and generating an acoustic line signal with respect to each of measurement points of the target area, by performing processing for specifying one measurement point signal for each of the transmission events and performing weighted delay-and-summing of measurement point signals specified for the respective transmission events, the processing including identification of one of the transducer elements that is located on a straight line passing through the measurement point and the focal point and specification of, as the measurement point signal, a receive signal corresponding to the identified transducer element from among the sequence of receive signals for the identified transducer element.

According to the above structure or method, it is possible to greatly reduce computation amount by refraining from performing delay-and-summing for each transmission event while enjoying an effect of an improvement in spatial resolution and S/N ratio achieved by virtual transmission focusing using a synthetic aperture method.

(2) Also, in the ultrasound signal processing device in the above item (1), the ultrasound signal processing circuitry may be further configured to operate as: a delay-and-sum synthesizer that generates a synthesized acoustic line signal with respect to each of measurement points of the target area, by performing processing for specifying one measurement point signal for each of the transmission events, performing weighted delay-and-summing of measurement point signals specified for the respective transmission events to generate a second acoustic line signal, and synthesizing the second acoustic line signal and the acoustic line signal generated by the delay-and-sum calculator to generate a synthesized acoustic line signal, the processing including identification of one of the transducer elements that is located on a straight line passing through the measurement point and is perpendicular to a direction in which the transducer elements are arrayed and specification of, as the measurement point signal, a receive signal corresponding to the identified transducer element from among the sequence of receive signals for the identified transducer element.

According to the above structure, it is possible to further improve S/N ratio of acoustic line signals, in the ultrasound signal processing device in the above item (1), by increasing the number of measurement point signals and performing delay-and-summing of two acoustic line signals acquired with respect to different receive transducer elements for one transmission event.

(3) Also, in the ultrasound signal processing device in the above item (1) or (2), the delay-and-sum calculator may use, as a transmission time being a time amount required for transmitted ultrasound to arrive at each of the measurement points, a total of a first time amount and a second time amount for the measurement point located at a depth no smaller than a focal depth where ultrasound converges inside the subject, and a difference calculated by subtracting the second time amount from the first time amount for the measurement point located at a depth smaller than the focal depth, the first time amount being a time amount required for ultrasound transmitted from a series of transmission transducer elements, among the transducer elements of the ultrasound probe, to arrive at a reference point, the second time amount being a time amount required for transmitted ultrasound to arrive at the measurement point from the reference point.

According to the above structure, it is possible to specify measurement point signals with high precision, thereby increasing an effect of an improvement in spatial resolution and S/N ratio of frame acoustic line signals.

(4) Also, in the ultrasound signal processing device in the above items (1)-(3), the ultrasound signal processing circuitry may be further configured to operate as: a second delay-and-sum calculator that generates, for each of the transmission events, a sub-frame acoustic line signal with respect to each of a plurality of second measurement points of a second target area, by performing delay-and-summing of receive signals included in the sequences of receive signals generated based on ultrasound reflection acquired from the second measurement point; and a synthesizer that synthesizes the acoustic line signal generated by the delay-and-sum calculator and the sub-frame acoustic line signals for the respective transmission events to generate a frame acoustic line signal.

According to the above structure, it is possible to further improve spatial resolution and S/N ratio by a combination of acoustic line signals acquired by conventional receive beam forming according to which delay-and-summing for each transmission event and acoustic line signals acquired without performing delay-and-summing for each transmission event.

(5) Also, in the ultrasound signal processing device in the above item (4), the second target area may be composed of one or more straight lines that pass through the focal point or a focal area and are perpendicular to a direction in which the transducer elements are arrayed, and the second delay-and-sum calculator may calculate a transmission time with respect to each of the second measurement points by dividing a depth of the second measurement point by an ultrasound velocity in the subject, the transmission time being a time amount required for transmitted ultrasound to arrive at the second measurement point.

According to the above structure, computation amount by the ultrasound signal processing device does not greatly increase due to a small computation amount by the second delay-and-sum calculator. Meanwhile, the delay-and-sum calculator and the second delay-and-sum calculator perform different computations, and accordingly can complement each other in terms of quality. This improves spatial resolution and signal S/N ratio.

(6) Also, in the ultrasound signal processing device in the above item (4), the second target area may be part or all of the target area, and the second delay-and-sum calculator may use, as a transmission time being a time amount required for transmitted ultrasound to arrive at each of the second measurement points, a total of a third time amount and a fourth time amount for the second measurement point located at a depth no smaller than a focal depth where ultrasound converges inside the subject, and a difference calculated by subtracting the fourth time amount from the third time amount for the second measurement point located at a depth smaller than the focal depth, the third time amount being a time amount required for ultrasound transmitted from a series of transmission transducer elements, among the transducer elements of the ultrasound probe, to arrive at a reference point, the fourth time amount being a time amount required for transmitted ultrasound to arrive at the second measurement point from the reference point.

According to the above structure, it is possible to improve spatial resolution and S/N ratio of frame acoustic line signals by sub-frame acoustic line signals acquired using a conventional synthetic aperture method.

(7) Also, in the ultrasound signal processing device in the above item (6), the second target area may be part of the target area, and may be smaller than the target area in terms of at least one of width and measurement point density in a direction in which the transducer elements are arrayed.

According to the above structure, it is possible to reduce computation amount by the second delay-and-sum calculator and complement quality deterioration due to a decrease in computation amount with an acoustic line signal generated by the second delay-and-sum calculator.

(8) Also, in the ultrasound signal processing device in the above items (4)-(7), the synthesizer may synthesize the acoustic line signal and the sub-frame acoustic line signals at a ratio varying depending on weighting based on a distance between each of the measurement points and a focal depth where ultrasound converges inside the subject.

According to the above structure, assume a case for example where spatial resolution or S/N ratio of the sub-frame acoustic line signals generated by the second delay-and-sum calculator depends on a distance from the focal depth. With respect to a depth corresponding to high spatial resolution or S/N ratio of the sub-frame acoustic line signals, it is possible to maintain quality of the sub-frame acoustic line signals. With respect to a depth corresponding to not high spatial resolution or S/N ratio, it is possible to improve quality of frame acoustic line signals by an acoustic line signal generated by the delay-and-sum calculator.

(9) Also, in the ultrasound signal processing device in the above items (4)-(7), the synthesizer may synthesize the acoustic line signal and the sub-frame acoustic line signals at a ratio varying depending on weighting based on the number of synthesized sub-frame acoustic line signals for each of the measurement points.

According to the above structure, it is possible to improve spatial resolution and S/N ratio by an acoustic line signal generated by the delay-and-sum calculator, with respect to for example an area with a small overlap count of sub-frame acoustic line signals.

(10) One aspect of the present invention is an ultrasound diagnostic device comprises an ultrasound probe and the ultrasound signal processing device in the above items (1)-(9).

An ultrasound signal processing device pertaining to the present invention, an ultrasound diagnostic device pertaining to the present invention, and an ultrasound signal processing method pertaining to the present invention are useful in improving the performance of conventional ultrasound diagnostic devices, and in particular, are useful in reducing computation device cost and in improving frame rate through reduction in computation load. In addition, the present invention, as well as being applicable to ultrasound, is also applicable for example to sensors having array elements.

Although embodiments of the present invention have been described and illustrated in detail, it is clearly understood that the same is by way of illustration and example only and not limitation; the scope of the present invention should be interpreted by terms of the appended claims.

What is claimed is:

1. An ultrasound signal processing device that performs multiple transmission events of transmitting converging ultrasound beams to a subject by using an ultrasound probe having multiple transducer elements, and that receives ultrasound reflection from the subject for each of the transmission events to generate sequences of receive signals, and synthesizes sequences of receive signals generated for the respective transmission events to generate an acoustic line signal,
the ultrasound signal processing device comprising ultrasound signal processing circuitry configured to operate as:
a transmitter that varies a focal point defining a position where ultrasound beams converge between a plurality of transmission events and performs each of the transmission events by causing the ultrasound probe to transmit ultrasound beams directed to an inside of the subject;
a receiver that generates, for each of the transmission events, sequences of receive signals for the respective transducer elements of the ultrasound probe based on ultrasound reflection that the ultrasound probe receives from a target area of the subject; and
a delay-and-sum calculator that generates an acoustic line signal with respect to each of measurement points of the target area, by performing processing for specifying one measurement point signal for each of the transmission events and performing weighted delay-and-summing of measurement point signals specified for the respective transmission events, the processing including identification of one of the transducer elements that is located on a straight line passing through the measurement point and the focal point and specification of, as the measurement point signal, a receive signal corresponding to the identified transducer element from among the sequence of receive signals for the identified transducer element.

2. The ultrasound signal processing device of claim 1, wherein
the ultrasound signal processing circuitry is further configured to operate as:
a delay-and-sum synthesizer that generates a synthesized acoustic line signal with respect to each of measurement points of the target area, by performing processing for specifying one measurement point signal for each of the transmission events, performing weighted delay-and-summing of measurement point signals specified for the respective transmission events to generate a second acoustic line signal, and synthesizing the second acoustic line signal and the acoustic line signal generated by the delay-and-sum calculator to generate a synthesized acoustic line signal, the processing including identification of one of the transducer elements that is located on a straight line passing through the measurement point and is perpendicular to a direction in which the transducer elements are arrayed and specification of, as the measurement point signal, a receive signal corresponding to the identified transducer element from among the sequence of receive signals for the identified transducer element.

3. The ultrasound signal processing device of claim 1, wherein
the delay-and-sum calculator uses, as a transmission time being a time amount required for transmitted ultrasound to arrive at each of the measurement points, a total of a first time amount and a second time amount for the measurement point located at a depth no smaller than a focal depth where ultrasound converges inside the subject, and a difference calculated by subtracting the second time amount from the first time amount for the measurement point located at a depth smaller than the focal depth, the first time amount being a time amount required for ultrasound transmitted from a series of transmission transducer elements, among the transducer elements of the ultrasound probe, to arrive at a reference point, the second time amount being a time amount required for transmitted ultrasound to arrive at the measurement point from the reference point.

4. The ultrasound signal processing device of claim 1, wherein
the ultrasound signal processing circuitry is further configured to operate as:
a second delay-and-sum calculator that generates, for each of the transmission events, a sub-frame acoustic line signal with respect to each of a plurality of second measurement points of a second target area, by performing delay-and-summing of receive signals included in the sequences of receive signals generated based on ultrasound reflection acquired from the second measurement point; and
a synthesizer that synthesizes the acoustic line signal generated by the delay-and-sum calculator and the sub-frame acoustic line signals for the respective transmission events to generate a frame acoustic line signal.

5. The ultrasound signal processing device of claim 4, wherein
the second target area is composed of one or more straight lines that pass through the focal point or a focal area and are perpendicular to a direction in which the transducer elements are arrayed, and
the second delay-and-sum calculator calculates a transmission time with respect to each of the second measurement points by dividing a depth of the second measurement point by an ultrasound velocity in the subject, the transmission time being a time amount required for transmitted ultrasound to arrive at the second measurement point.

6. The ultrasound signal processing device of claim 4, wherein
the second target area is part or all of the target area, and
the second delay-and-sum calculator uses, as a transmission time being a time amount required for transmitted ultrasound to arrive at each of the second measurement points, a total of a third time amount and a fourth time amount for the second measurement point located at a depth no smaller than a focal depth where ultrasound converges inside the subject, and a difference calculated by subtracting the fourth time amount from the third time amount for the second measurement point located at a depth smaller than the focal depth, the third time amount being a time amount required for ultrasound transmitted from a series of transmission transducer elements, among the transducer elements of the ultrasound probe, to arrive at a reference point, the fourth time amount being a time amount required for transmitted ultrasound to arrive at the second measurement point from the reference point.

7. The ultrasound signal processing device of claim 6, wherein
the second target area is part of the target area, and is smaller than the target area in terms of at least one of width and measurement point density in a direction in which the transducer elements are arrayed.

8. The ultrasound signal processing device of claim 4, wherein
the synthesizer synthesizes the acoustic line signal and the sub-frame acoustic line signals at a ratio varying depending on weighting based on a distance between each of the measurement points and a focal depth where ultrasound converges inside the subject.

9. The ultrasound signal processing device of claim 4, wherein
the synthesizer synthesizes the acoustic line signal and the sub-frame acoustic line signals at a ratio varying depending on weighting based on the number of synthesized sub-frame acoustic line signals for each of the measurement points.

10. An ultrasound diagnostic device that performs multiple transmission events of transmitting converging ultrasound beams to a subject, and that receives ultrasound reflection from the subject for each of the transmission events to generate sequences of receive signals, and synthesizes sequences of receive signals generated for the respective transmission events to generate an acoustic line signal,
the ultrasound diagnostic device comprising:
an ultrasound probe having multiple transducer elements; and
ultrasound signal processing circuitry configured to operate as:
a transmitter that varies a focal point defining a position where ultrasound beams converge between a plurality of transmission events and performs each of the transmission events by causing the ultrasound probe to transmit ultrasound beams directed to an inside of the subject;
a receiver that generates, for each of the transmission events, sequences of receive signals for the respective transducer elements of the ultrasound probe based on ultrasound reflection that the ultrasound probe receives from a target area of the subject; and
a delay-and-sum calculator that generates an acoustic line signal with respect to each of measurement points of the target area, by performing processing for specifying one measurement point signal for each of the transmission events and performing weighted delay-and-summing of measurement point signals specified for the respective transmission events, the processing including identification of one of the transducer elements that is located on a straight line passing through the measurement point and the focal point and specification of, as the measurement point signal, a receive signal corresponding to the identified transducer element from among the sequence of receive signals for the identified transducer element.

11. An ultrasound signal processing method in which multiple transmission events of transmitting converging ultrasound beams to a subject are performed by using an ultrasound probe having multiple transducer elements, and in which ultrasound reflection is received from the subject for each of the transmission events to generate sequences of receive signals, and sequences of receive signals generated for the respective transmission events are synthesized to generate an acoustic line signal, the ultrasound signal processing method comprising:
varying a focal point defining a position where ultrasound beams converge between a plurality of transmission events and performing each of the transmission events by causing the ultrasound probe to transmit ultrasound beams directed to an inside of the subject;
generating, for each of the transmission events, sequences of receive signals for the respective transducer elements of the ultrasound probe based on ultrasound reflection that the ultrasound probe receives from a target area of the subject; and
generating an acoustic line signal with respect to each of measurement points of the target area, by performing processing for specifying one measurement point signal for each of the transmission events and performing weighted delay-and-summing of measurement point signals specified for the respective transmission events, the processing including identification of one of the transducer elements that is located on a straight line passing through the measurement point and the focal point and specification of, as the measurement point signal, a receive signal corresponding to the identified transducer element from among the sequence of receive signals for the identified transducer element.
